(19) United States
(12) Patent Application Publication
(10) Pub. No.: US 2026/0356952 A1
(43) Pub. Date: Nov. 12, 2026

(54) STABLE FORMULATIONS OF PROGRAMMED DEATH RECEPTOR 1 (PD-1) ANTIBODIES AND HYALURONIDASE VARIANTS AND FRAGMENTS THEREOF AND METHODS OF USE THEREOF

(71) Applicant: MERCK SHARP & DOHME LLC, Rahway, NJ (US)

(72) Inventors: Yogita KRISHNAMACHARI, Rahway, NJ (US); Sachin MITTAL, Rahway, NJ (US); Sahil S. SANGANI, Rahway, NJ (US); William P. FORREST, Rahway, NJ (US); Yongchao SU, Rahway, NJ (US); Xi ZHAO, Rahway, NJ (US); Katelyn Jean SMITH, Rahway, NJ (US)

(73) Assignee: MERCK SHARP & DOHME LLC, Rahway, NJ (US)

(21) Appl. No.: 19/741,007

(22) Filed: Jul. 14, 2026

Related U.S. Application Data
(63) Continuation of application No. 18/246,467, filed on Sep. 23, 2021, now abandoned.
(60) Provisional application No. 63/082,888, filed on Sep. 24, 2020.

(51) Int. Cl. C07K 16/28; A61K 9/19; A61K 38/47; A61K 39/00; A61K 47/18; A61K 47/26; C12N 9/26

(52) U.S. Cl. CPC C07K 16/2818; A61K 9/19; A61K 38/47; A61K 47/183; A61K 47/26; C12N 9/2474; A61K 2039/545; C12Y 302/01035

(57) ABSTRACT

The invention relates to stable formulations of antibodies against human programmed death receptor PD-1, or antigen binding fragments thereof and a PH20 variant or fragment thereof. The invention further provides methods for treating various cancers with formulations of the invention. In some embodiments of the methods of the invention, the formulations are administered to a subject by subcutaneous administration.

### CROSS-REFERENCE TO RELATED APPLICATIONS

This application is a continuation application of U.S. patent application Ser. No. 18/246,467, filed Mar. 23, 2023, which is a national stage application of International Patent Application No. PCT/US2021/051641, filed Sep. 23, 2021, which claims the benefit of U.S. Provisional Patent Application Ser. No. 63/082,888, filed Sep. 24, 2020, each of which is incorporated by reference in its entirety herein.

### SEQUENCE LISTING

The instant application contains a Sequence Listing which has been submitted electronically. The electronic sequence listing is named 25106-US-CNT.xml; created on Jul. 10, 2026, Size: 33,788 bytes and is herein incorporated by reference in its entirety.

### FIELD OF THE INVENTION

The invention relates to stable formulations comprising antibodies or antigen binding fragments thereof that bind to human programmed death receptor 1 (PD-1) and hyaluronic acid-hydrolyzing enzyme and variants thereof. Also provided are methods of treating various cancers and chronic infections with the formulations of the invention.

### BACKGROUND OF THE INVENTION

Immune checkpoint therapies targeting the programmed death receptor-1 (PD-1) axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33). The interaction of the PD-1 receptor on T-cells with its ligands, PD-L1 and PD-L2, on tumor and immune infiltrating cells regulates T-cell mediated immune responses and may play a role in immune escape by human tumors (Pardoll D M. Nat Rev Cancer 2012, 12: 252-64). Binding of PD-1 to either of its ligands results in delivery of an inhibitory stimulus to the T cell. Immune therapies targeting the PD-1 axis include monoclonal antibodies directed to the PD-1 receptor (KEYTRUDA™ (pembrolizumab), Merck and Co., Inc., Kenilworth, NJ and OPDIVO™ (nivolumab), Bristol-Myers Squibb, Princeton, NJ) and also those that bind to the PD-L1 ligand (MPDL3280A; TECENTRIQ™ (atezolizumab), Genentech, San Francisco, CA). Both therapeutic approaches have demonstrated anti-tumor effects in numerous cancer types.

Hyaluronidases are enzymes that degrade hyaluronic acid present in the extracellular matrix. It is known that there are six types of hyaluronidases in humans: Hyall, Hyal2, Hyal3, Hyal4, HyalPS1, and PH20/SPAM1. PH20/SPAM1 (hereinafter referred to as PH20) is expressed in the sperm plasma membrane and the acrosomal membrane.

Hyaluronidase hydrolyzes hyaluronic acid, thereby reducing the viscosity of hyaluronic acid in the extracellular matrix and increasing the permeability thereof into tissue (skin). The subcutaneous area of the skin has a neutral pH of about 7.0 to 7.5. Thus, among the various types of hyaluronidases, PH20 is widely used (Bookbinder et al., 2006). In examples in which PH20 is used, PH20 is often co-administered with an antibody therapeutic agent which is injected subcutaneously (Bookbinder et al., 2006).

The stability of a formulation of an antibody and enzyme is complex and confounded by multiple factors such as function of stability of the individual enzyme or antibody in the co-formulation matrix, impact of the presence of the additional excipients, and specific interaction of the antibody and enzyme. Often times, high concentration of antibody formulated with an enzyme, may contribute to other properties of the product which would be undesirable, e.g. low injectability due to increased viscosity and higher than physiological osmolality and increased aggregation. As a consequence, the need exists for stable formulations of an anti-PD-1 antibody and PH20 or PH20 variants for subcutaneous administration. Such stable formulations will preferably exhibit stability over months to years under conditions typical for storage of drugs for self-administration, i.e. at refrigerator temperature in a syringe, container or other device, resulting in a long shelf-life for the corresponding drug product.

### SUMMARY OF THE INVENTION

The invention provides a formulation, comprising:
a) about 20 mg/mL to about 200 mg/mL of an anti-human PD-1 antibody, or antigen binding fragment thereof, b) about 0.0009-0.050 mg/ml of a PH20 variant or fragment thereof, c) a buffer; d) a non-reducing dissacharide; e) a non-ionic surfactant; and, optionally f) an anti-oxidant.

In one embodiment, the formulation comprises:
a) about 20 mg/mL to about 200 mg/mL of an anti-human PD-1 antibody, or antigen binding fragment thereof,
b) about 0.0009-0.050 mg/ml of a PH20 variant or fragment thereof,
c) about 5 mM to about 20 mM buffer;
d) about 3% to about 10% weight/volume (w/v) of a non-reducing dissacharide selected from the group consisting of sucrose and trehalose;
e) about 0.005% to about 0.10% non-ionic surfactant; and, optionally
f) about 1 mM to about 30 mM anti-oxidant.

Surprisingly, certain embodiments of the formulations of the invention have increased hyaluronidase activity of the PH20 variant or fragment thereof compared to the corresponding PH20 variant or fragment thereof alone in the same formulation after 1 or 3 months storage at 25 °C; after 6 months at 5 °C and after 3 months at 25 °C under stainless steel stress; and under light stress. The invention can be a liquid formulation or a liquid formulation that is reconstituted from a lyophilized formulation.

In specific embodiments of the invention, the anti-PD-1 antibody is pembrolizumab or an antigen binding fragment of pembrolizumab. In specific embodiments of the invention, the PH20 variant or fragment is PH20 variant fragment 2 set forth in the amino acid sequence of SEQ ID NO: 23.

Also provided herein are methods of treating cancer and methods of treating chronic infection in a human patient in need thereof comprising: administering an effective amount of the formulations of the invention to the patient.

### BRIEF DESCRIPTION OF THE DRAWINGS

FIG. 1. Temperature-dependent viscosity profiles at 5 and 25 °C for formulations with AK03 (pembrolizumab only at 100 mg/ml) and AK10-AK15 (pembrolizumab and PH20 variant fragment 2 at different concentrations)

FIG. 2. % High Molecular Weight Species (HMWS) as measured by UP-SEC of formulations AK03 (pembrolizumab only at 100 mg/ml) and AK10-AK15 (pembrolizumab and PH20 variant fragment 2 at different concentrations) at 5, 25 and 40 °C at initial, 1 month, 3 months and 6 months, respectively.

FIG. 3. Acidic Variants as measured by HP-IEX of formulations AK03 (pembrolizumab only at 100 mg/ml) and AK10-AK15 (pembrolizumab and PH20 variant fragment 2 at different concentrations) at 5, 25 and 40 °C at initial, 1 month, 3 months and 6 months, respectively.

FIG. 4. Main Variants as measured by HP-IEX of formulations AK03 (pembrolizumab only at 100 mg/ml) and AK10-AK15 (pembrolizumab and PH20 variant fragment 2 at different concentrations) at 5, 25 and 40 °C at initial, 1 month, 3 months and 6 months, respectively.

FIG. 5. Basic Variants as measured by HP-IEX of formulations AK03 (pembrolizumab only at 100 mg/ml) and AK10-AK15 (pembrolizumab and PH20 variant fragment 2 at different concentrations) at 5, 25 and 40 °C at initial, 1 month, 3 months and 6 months, respectively.

FIG. 6. Met[105] Oxidation species (the % of pre-peak 1+2) as measured by HP-HIC in pembrolizumab of formulations AK03 (pembrolizumab only at 100 mg/ml) and AK10-AK15 (pembrolizumab and PH20 variant fragment 2 at different concentrations) at 5, 25 and 40 °C at initial, 1 month, 3 months and 6 months, respectively.

FIG. 7. Example activity assay calibration curve. The plot was fit as a second-order polynomial and the resulting equation of the fit was used to determine hyaluronidase activity of the activity standards and test samples.

FIG. 8. Activity of formulations and PH20 variant fragment 2 control. Activity after storage at 5 °C for three (gray bars) and six months (white bars) is comparable to T0 samples (black bars).

FIG. 9. Activity of formulations and PH20 variant fragment 2 control after storage at 5 °C (gray bars) and 25 °C (checkered bars) for three months. Only PH20 variant fragment 2 control samples (AK05 and AK06) show decreased activity upon 25 °C storage.

FIG. 10. DSC thermogram of Pembrolizumab and PH20 variant fragment 2.

FIG. 11. Enzyme activity with SS stress staged up 6 months at 5 °C.

FIG. 12. Enzyme activity of Pembrolizumab+PH20 variant fragment 2 samples after SS stress followed by incubation for 3 months at 25 °C.

FIG. 13. PH20 variant fragment 2 activity in the presence of Pembrolizumab (white bars) or viscosity surrogate (black bars) under light stress. LS stands for light stress; LS DC stands for the dark control for light stress, where vials were wrapped with aluminum foil and placed in the light chamber together with those stressed by light.

FIG. 14. Retention of PH20 variant fragment 2 enzyme activity with pembrolizumab across a range of excipient concentrations compared to PH20 variant fragment 2 alone (AK05). Data at initial time point, 1 and 3 months at 25 °C.

FIG. 15. Retention of PH20 variant fragment 2 enzyme activity across a range of antibody:enzyme ratios compared to PH20 variant fragment 2 alone (AK05). Data at initial time point, 1 and 3 months at 25 °C.

FIG. 16. Impact of pembrolizumab concentration on PH20 variant fragment 2 enzyme activity upon thermal stress.

FIG. 17. Impact of pH on PH20 variant fragment 2 activity. Data at initial time point, 1 and 3 months at 25 °C.

FIG. 18. Example activity assay calibration curve. A linear fit was applied to the data and the resulting equation of the fit was used to determine enzymatic activity of the activity standards and test samples.

### DETAILED DESCRIPTION OF THE INVENTION

The invention provides stable formulations or compositions comprising an anti-PD-1 antibody, or antigen binding fragment thereof that binds to human PD-1 and a PH20 variant or fragment thereof, which are useful for methods of treatment of cancer or an immune disorder or immune condition for administration to a patient in need thereof. In certain embodiments of the invention, the anti-PD-1 antibody is pembrolizumab or an antigen binding fragment of pembrolizumab. In certain embodiments of the invention, the formulations of the invention are for subcutaneous administrations. Surprisingly, the above formulations and compositions have increased hyaluronidase activity of the PH20 variant or fragment thereof compared to the corresponding PH20 variant or fragment thereof alone in the same formulation after 1 or 3 months storage at 25 °C. The formulations of the invention are useful for subcutaneous delivery to a patient in need thereof.

Certain embodiments of the formulations of the invention maintain the low methionine-105 oxidation levels (which is located in CDR3 of the heavy chains) of pembrolizumab compared to the same formulation without the PH20 variant or fragment thereof at 5 or 25 °C at 6 months. Major degradation pathways of pembrolizumab include oxidation of methionine 105 (Met105) in the heavy chain CDR upon peroxide stress and oxidation of Met105 and Fc methionine residues when exposed to light. Reduction in affinity to PD-1 was observed for peroxide stressed samples by Surface Plasmon Resonance (SPR). An exposed methionine residue or a methionine residue in the CDR of an antibody has the potential of impacting the biological activity of the antibody through oxidation.

Certain embodiments of the formulations of the invention maintain the low level of aggregation of pembrolizumab compared to the same formulation without the PH20 variant or fragment thereof at 5-40 °C at 6 months.

### DEFINITIONS AND ABBREVIATIONS

As used throughout the specification and appended claims, the following abbreviations apply:

| | |
|---|---|
| API | active pharmaceutical ingredient |
| CDR | complementarity determining region in the immunoglobulin |
| | variable regions |
| CE-SDS | capillary electrophoresis-sodium dodecyl sulfate |
| CHO | Chinese hamster ovary |
| CI | confidence interval |
| DS | drug substance |
| EC50 | concentration resulting in 50% efficacy or binding |
| ELISA | enzyme-linked immunosorbant assay |
| FFPE | formalin-fixed, paraffin-embedded |
| FR | framework region |
| HC | heavy chain |
| HNSCC | head and neck squamous cell carcinoma |
| HP-HIC | high performance hydrophobic interaction chromatography |
| HP-IEX | high performance ion-exchange chromatography |
| HP-SEC | high performance size exclusion chromatography |
| IC50 | concentration resulting in 50% inhibition |
| IgG | immunoglobulin G |
| IHC | immunohistochemistry or immunohistochemical |
| mAb | monoclonal antibody |
| NCBI | National Center for Biotechnology Information |
| NSCLC | non-small cell lung cancer |
| PCR | polymerase chain reaction |
| PD-1 | programmed death 1 (a.k.a. programmed cell death-1 and |
| | programmed death receptor 1) |
| PD-L1 | programmed cell death 1 ligand 1 |
| PD-L2 | programmed cell death 1 ligand 2 |
| PS80 or | polysorbate 80 |
| PS-80 | |
| SWFI | sterile water for injection |
| TNBC | triple negative breast cancer |
| VH | immunoglobulin heavy chain variable region |
| VK | immunoglobulin kappa light chain variable region |
| VL | immunoglobulin light chain variable region |
| VP-DSC | Valerian-Plotnikov differential scanning calorimetry |
| v/V | volume per volume |
| WFI | water for injection |
| w/V | weight per volume |

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” a cancer as used herein means to administer a formulation of the invention to a subject having an immune condition or cancerous condition, or diagnosed with a cancer or pathogenic infection (e.g. viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating, abrogating the clinical manifestations of PD-1-related disease, reducing the severity or duration of the clinical symptoms of PD-1-related disease such as cancer, prolonging the survival of a patient relative to the expected survival in a similar untreated patient, inducing complete or partial remission of a cancerous condition or other PD-1 related disease.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by administration of a formulation of the invention is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the formulations, treatment methods, and uses of the invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the formulations or compositions of the invention, most preferably a human. In some embodiments, the patient is an adult patient. In other embodiments, the patient is a pediatric patient. Those “in need of treatment” include those patients that may benefit from treatment with the formulations or compositions of the invention, e.g. a patient suffering from cancer or an immune condition.

The term “antibody” refers to any form of antibody that exhibits the desired biological activity. Thus, it is used in the broadest sense and specifically covers, but is not limited to, monoclonal antibodies (including full length monoclonal antibodies), polyclonal antibodies, humanized, fully human antibodies, and chimeric antibodies.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

An antibody or antigen-binding fragment that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “pharmaceutically effective amount” or “effective amount” means an amount whereby sufficient therapeutic composition or formulation is introduced to a patient to treat a diseased or condition. One skilled in the art recognizes that this level may vary according the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%.

As used herein, “x % (w/v)” is equivalent to x g/100 ml (for example, 5% w/v equals 50 mg/ml).

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

A “chemotherapeutic agent” is a chemical compound useful in the treatment of cancer. Anti-PD-1 antibodies can be used with any one or more suitable chemotherapeutic agent. Examples of such chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammalI and calicheamicin phiIl, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

“Chothia” means an antibody numbering system described in Al-Lazikani et al., JMB 273:927-948 (1997).

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

A “growth inhibitory agent” when used herein refers to a compound or composition which inhibits growth of a cell, especially cancer cell over expressing any of the genes identified herein, either in vitro or in vivo. Thus, the growth inhibitory agent is one which significantly reduces the percentage of cells over expressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine) taxanes, and topo II inhibitors such as doxorubicin, epirubicin, daunorubicin, and etoposide. Those agents that arrest G1 also spill over into S-phase arrest, for example, DNA alkylating agents such as dacarbazine, mechlorethamine, and cisplatin. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995).

The terms “PD-1 binding fragment,” “antigen binding fragment thereof,” “binding fragment thereof” or “fragment thereof” encompass a fragment or a derivative of an antibody that still substantially retains its biological activity of binding to antigen (human PD-1) and inhibiting its activity (e.g., blocking the binding of PD-1 to PDL1 and PDL2). Therefore, the term “antibody fragment” or PD-1 binding fragment refers to a portion of a full length antibody, generally the antigen binding or variable region thereof. Examples of antibody fragments include Fab, Fab′, F(ab′)₂, and Fv fragments. Typically, a binding fragment or derivative retains at least 10% of its PD-1 inhibitory activity. In some embodiments, a binding fragment or derivative retains at least 25%, 50%, 60%, 70%, 80%, 90%, 95%, 99% or 100% (or more) of its PD-1 inhibitory activity, although any binding fragment with sufficient affinity to exert the desired biological effect will be useful. In some embodiments, an antigen binding fragment binds to its antigen with an affinity that is at least two fold greater, preferably at least ten times greater, more preferably at least 20-times greater, and most preferably at least 100-times greater than the affinity with unrelated antigens. In one embodiment the antibody has an affinity that is greater than about 109 liters/mol, as determined, e.g., by Scatchard analysis. Munsen et al. (1980) Analyt. Biochem. 107:220-239. It is also intended that a PD-1 binding fragment can include variants having conservative amino acid substitutions that do not substantially alter its biologic activity.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

The antibodies of the invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

“Fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only. A fully human antibody may be generated in a human being, in a transgenic animal having human immunoglobulin germline sequences, by phage display or other molecular biological methods.

“Hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain as measured by the Kabat numbering system (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J Mol. Biol. 196: 901-917). As used herein, the term “framework” or “FR” residues refers to those variable domain residues other than the hypervariable region residues defined herein as CDR residues. CDR and FR residues are determined according to the standard sequence definition of Kabat. Kabat et al. (1987) Sequences of Proteins of Immunological Interest, National Institutes of Health, Bethesda Md.

“Conservatively modified variants” or “conservative substitution” refers to substitutions of amino acids that are known to those of skill in this art and may be made generally without altering the biological activity of the resulting molecule, even in essential regions of the polypeptide. Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In addition, those of skill in this art recognize that, in general, single amino acid substitutions in non-essential regions of a polypeptide do not substantially alter biological activity. See, e.g., Watson et al. (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J Mol. Biol. 222: 581-597, for example. See also Presta (2005) J. Allergy Clin. Immunol. 116:731.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Tumor Proportion Score (TPS)” refers to the percentage of tumor cells expressing PD-L1 on the cell membrane at any intensity (weak, moderate or strong). Linear partial or complete cell membrane staining is interpreted as positive for PD-L1.

“Mononuclear inflammatory density score (MIDS)” refers to the ratio of the number of PD-L1 expressing mononuclear inflammatory cells (MIC) infiltrating or adjacent to the tumor (small and large lymphocytes, monocytes, and macrophages within the tumor nests and the adjacent supporting stroma) compared to the total number of tumor cells. The MIDS is recorded at a scale from 0 to 4 with 0=none; 1=present, but less than one MIC for every 100 tumor cells (<1%); 2=at least one MIC for every 100 tumor cells, but less than one MIC per 10 tumor cells (1-9%); 3=at least one MIC for every 10 tumor cells, but fewer MIC's than tumor cells (10-99%); 4=at least as many MIC's as tumor cells (>100%).

“Combined positive score (CPS)” refers to the ratio of the number of PD-L1 positive tumor cells and PD-L1 positive mononuclear inflammatory cells (MIC) within the tumor nests and the adjacent supporting stroma (numerator) compared to the total number of tumor cells (denominator; i.e., the number of PD-L1 positive and PD-L1 negative tumor cells). PD-L1 expression at any intensity is considered positive, i.e., weak (1+), moderate (2+), or strong (3+).

“PD-L1 expression positive” refers to a Tumor Proportion Score, Mononuclear Inflammatory Density Score or Combined Positive Score of at least 1%; AIS is ≥5; or elevated level of PD-L1 expression (protein and/or mRNA) by malignant cells and/or by infiltrating immune cells within a tumor compared to an appropriate control.

“Microsatellite instability (MSI)” refers to the form of genomic instability associated with defective DNA mismatch repair in tumors. See Boland et al., Cancer Research 58, 5258-5257, 1998. In one embodiment, MSI analysis can be carried out using the five National Cancer Institute (NCI) recommended microsatellite markers: BAT25 (GenBank accession no. 9834508), BAT26 (GenBank accession no. 9834505), D5S346 (GenBank accession no. 181171), D2S123 (GenBank accession no. 187953), D17S250 (GenBank accession no. 177030). Additional markers for example, BAT40, BAT34C4, TGF-3-RII and ACTC can be used. Commercially available kits for MSI analysis include, for example, the Promega MSI multiplex PCR assay, FoundationOne® CDx (F1CDx) next generation sequencing based in vitro diagnostic device using DNA isolated from formalin-fixed, paraffin-embedded (FFPE) tumor tissue specimens.

“High frequency microsatellite instability” or “microsatellite instability-high (MSI-H)” refers to if two or more of the five NCI markers indicated above show instability or ≥30-40% of the total markers demonstrate instability (i.e. have insertion/deletion mutations).

“Non-MSI-H cancer” as used herein refers to microsatellite stable (MSS) and low frequency MSI (MSI-L) cancer.

“Microsatellite Stable (MSS)” refers to if none of the five NCI markers indicated above show instability (i.e. have insertion/deletion mutations).

“Proficient mismatch repair (pMMR) cancer” refers to normal expression of MMR proteins (MLH1, PMS2, MSH2, and MSH6) in tumor specimen by IHC. Commercially available kits for MMR analysis include the Ventana MMR IHC assay.

“Mismatch repair deficient (dMMR) cancer” refers to low expression of one or more MMR protein(s) (MLH1, PMS2, MSH2, and MSH6) in a tumor specimen by IHC.

“Variable regions” or “V region” as used herein means the segment of IgG chains which is variable in sequence between different antibodies. It extends to Kabat residue 109 in the light chain and 113 in the heavy chain.

The term “buffer” encompasses those agents which maintain the solution pH of the formulations of the invention in an acceptable range, or, for lyophilized formulations of the invention, provide an acceptable solution pH prior to lyophilization.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

The term “pharmaceutical formulation” refers to preparations which are in such form as to permit the active ingredients to be effective, and which contains no additional components which are toxic to the subjects to which the formulation would be administered. The term “formulation” and “pharmaceutical formulation” are used interchangeably throughout.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

A “reconstituted” formulation is one that has been prepared by dissolving a lyophilized protein formulation in a diluent such that the protein is dispersed in the reconstituted formulation. The reconstituted formulation is suitable for administration, e.g. parenteral or intravenous administration, and may optionally be suitable for subcutaneous administration.

“Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8 °C) for at least 12 months. In another embodiment, a stable formulation is a formulation with no significant changes observed at a refrigerated temperature (2-8 °C) for at least 18 months. In another embodiment, stable formulation is a formulation with no significant changes observed at room temperature (23-27 °C) for at least 3 months. In another embodiment, stable formulation is a formulation with no significant changes observed at room temperature (23-27 °C) for at least 6 months. In another embodiment, stable formulation is a formulation with no significant changes observed at room temperature (23-27 °C) for at least 12 months. In another embodiment, stable formulation is a formulation with no significant changes observed at room temperature (23-27 °C) for at least 18 months. The criteria for stability for an antibody formulation are as follows. Typically, no more than 10%, preferably 5%, of antibody monomer is degraded as measured by SEC-HPLC. Typically, the formulation is colorless, or clear to slightly opalescent by visual analysis. Typically, the concentration, pH and osmolality of the formulation have no more than +/−10% change. Potency is typically within 60-140%, preferably 80-120% of the control or reference. Typically, no more than 10%, preferably 5% of clipping of the antibody is observed, i.e., % low molecular weight species as determined, for example, by HP-SEC. Typically, no more than 10%, preferably no more than 5% of aggregation of the antibody is observed, i.e. % high molecular weight species as determined, for example, by HP-SEC.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay. Formulations of the invention include antibodies and fragments thereof that are biologically active when reconstituted or in liquid form.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

A “non-reducing dissacharide” is a dissacharide not capable of acting as a reducing agent because it does not contain or cannot be converted to contain a free aldehyde group or a free ketone group. Examples of non-reducing dissacharides include but are not limited to dissacharrides such as sucrose and trehalose.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 2. Pembrolizumab has been approved by the U.S. FDA as described in the Prescribing Information for KEYTRUDA™ (Merck & Co., Inc., Whitehouse Station, NJ USA; initial U.S. approval 2014).

As used herein, a “pembrolizumab variant” means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“PH 20” refers to the wild-type PH20 hyaluronidase of SEQ ID NO: 21.

“PH20 variant” as used herein is a variant of PH20 that has amino acid residue substitutions including M345T, S347T, M348K, K349E, L352Q, L353A, L354I, D355K, N356E, E359D, and 1361T in SEQ ID NO: 21.

A “PH20 variant fragment” or “PH20 variant fragment thereof” “or “fragment of a PH20 variant” is a PH20 variant that has either an N-terminus deletion of amino acid residues 1-36, 1-37, 1-38, 1-39, 1-40, 1-41, or 1-42 of SEQ ID NO: 21; and/or a C-terminus deletion of amino acid residues 455-509, 456-509, 457-509, 458-509, 459-509, 460-509, 461-509, 462-509, 463-509, 464-509, 465-509, 466-509, 467-509, 468-509, 469-509, 470-509, 471-509, 472-509, 473-509, 474-509, 475-509, 476-509, 477-509, 478-509, 479-509, 480-509, 481-509, 482-509, 483-509, 484-509, 485-509, 486-509, 487-509, 488-509, 489-509, 490-509, 491-509, 492-509, 493-509, 494-509, 495-509, 496-509, 497-509, 498-509, 499-509, 500-509, 501-509, 502-509, 503-509, 504-509, 505-509, 506-509, 507-509, 508-509, or 509, wherein the numbering is by reference to SEQ ID NO: 21.

“Unit” or “U” refers to One unit of Hyaluronidase activity: amount of PH20 variant or fragment thereof that causes a change in the optical density at 600 nm at conditions suitable for reaction of hyaluronic acid and the enzyme and calculated according to a calibration curve using an activity standard. An example of the assay is described in Example 4. Hyaluronic acid (HA) binds to albumin and the albumin-HA complex develops turbidity. When HA is hydrolyzed by hyaluronidase, turbidity of albumin-HA complex is reduced. As such, this assay measures turbidity to determine hyaluronidase enzyme activity of PH20 variants or fragments thereof. Hyaluronidase activity is based on the following reaction: Hyaluronic acid→Di- and monosaccharides+smaller hyaluronic acid fragments. One skilled in the art understands that the hyalurodindase activity in Units per mg of hyaluronidase can vary depending on the purity, manufacturing process etc. of the hyaluronidase. In one embodiment, 2000 U/ml of PH20 variant fragment 2 is about 0.012 mg/ml, 4000 U/ml of PH20 variant fragment 2 is about 0.024 mg/ml, and 5000 U/ml of PH20 variant fragment 2 is about 0.030 mg/ml.

### Formulations of the Invention

The invention includes various formulations of a PD-1 antibody, or antigen binding fragment thereof and a PH20 variant or fragment thereof, as described in more detail, infra. For example, the invention includes formulations comprising (i) an anti-PD-1 antibody or antigen binding fragment thereof and a PH20 variant or fragment thereof, (ii) a buffer (e.g., histidine or acetate), (iii) a non-reducing dissacharide (e.g., a non-reducing dissacharide such as sucrose or trehalose; (iv) a non-ionic surfactant (e.g., polysorbate 80); and (v) an antioxidant (e.g., methionine).

In one aspect, the invention provides a formulation comprising:
a) about 20 mg/mL to about 200 mg/mL of an anti-human PD-1 antibody, or antigen binding fragment thereof,
b) about 0.0009-0.035 mg/ml of PH20 variant or fragment thereof,
c) about 5 mM to about 20 mM buffer;
d) about 1% to about 10% weight/volume (w/v) of a non-reducing dissacharide selected from the group consisting of sucrose and trehalose;
e) about 0.001% to about 0.10% non-ionic surfactant; and, optionally
f) about 1 mM to about 30 mM anti-oxidant.

In another aspect, the invention provides a formulation comprising:
a) about 100 mg/mL to about 185 mg/mL of an anti-human PD-1 antibody, or antigen binding fragment thereof,
b) about 0.01-0.04 mg/ml of PH20 variant or fragment thereof,
c) about 5 mM to about 20 mM histidine buffer;
d) about 6% to about 8% w/v sucrose;
e) about 0.01% to about 0.04% w/v polysorbate 80; and optionally
f) about 5 mM to about 20 mM L-methionine, or a pharmaceutically acceptable salt thereof.

In another aspect, the invention provides a formulation comprising:
a) about 100 mg/mL to about 165 mg/mL of an anti-human PD-1 antibody, or antigen binding fragment thereof,
b) about 0.012-0.030 mg/ml of PH20 variant or fragment thereof,
c) about 5 mM to about 20 mM histidine buffer;
d) about 6% to about 8% w/v sucrose;
e) about 0.01% to about 0.04% w/v polysorbate 80; and optionally
f) about 5 mM to about 20 mM L-methionine, or a pharmaceutically acceptable salt thereof.

In a further aspect, the invention provides a formulation, comprising:
a) about 130 mg/mL of an anti-human PD-1 antibody, or antigen binding fragment thereof,
b) about 0.012 mg/ml of PH20 or PH20 variant or fragment thereof,
c) about 8 mM to about 12 mM histidine buffer;
d) optionally, about 5 mM to about 10 mM L-methionine, or a pharmaceutically acceptable salt thereof;
e) about 6% to about 8% w/v sucrose; and
f) 0.01% to about 0.04% w/v polysorbate 80.

In a further aspect, the invention provides a formulation comprising:
a) about 130 mg/mL of an anti-human PD-1 antibody, or antigen binding fragment thereof,
b) about 0.024 mg/ml of PH20 or PH20 variant or fragment thereof,
c) about 8 mM to about 12 mM histidine buffer;
d) optionally, about 5 mM to about 10 mM L-methionine, or a pharmaceutically acceptable salt thereof;
e) about 6% to about 8% w/v sucrose; and
f) 0.01% to about 0.04% w/v polysorbate 80.

In a further aspect, the invention provides a formulation comprising:
a) about 130 mg/mL of an anti-human PD-1 antibody, or antigen binding fragment thereof,
b) about 0.030 mg/ml of PH20 or PH20 variant or fragment thereof,
c) 8 mM to about 12 mM histidine buffer;
d) optionally, about 5 mM to about 10 mM L-methionine, or a pharmaceutically acceptable salt thereof;
e) about 6% to about 8% w/v sucrose; and
f) 0.01% to about 0.04% w/v polysorbate 80.

In a further aspect, the invention provides a formulation comprising:
a) about 165 mg/mL of an anti-human PD-1 antibody, or antigen binding fragment thereof,
b) about 0.012 mg/ml of PH20 or PH20 variant or fragment thereof,
c) about 8 mM to about 12 mM histidine buffer;
d) optionally, about 5 mM to about 10 mM L-methionine, or a pharmaceutically acceptable salt thereof;
e) about 6% to about 8% w/v sucrose; and
f) about 0.01% to about 0.04% w/v polysorbate 80.

In a further aspect, the invention provides a formulation comprising:
a) about 165 mg/mL of an anti-human PD-1 antibody, or antigen binding fragment thereof,
b) about 0.024 mg/ml of PH20 or PH20 variant or fragment thereof,
c) about 8 mM to about 12 mM histidine buffer;
d) optionally, about 5 mM to about 10 mM L-methionine, or a pharmaceutically acceptable salt thereof;
e) about 6% to about 8% w/v sucrose; and
f) about 0.01% to about 0.04% w/v polysorbate 80.

In a further aspect, the invention provides a formulation comprising:
a) about 165 mg/mL of an anti-human PD-1 antibody, or antigen binding fragment thereof,
b) about 0.030 mg/ml of PH20 or PH20 variant or fragment thereof,
c) about 8 mM to about 12 mM histidine buffer;
d) optionally, about 5 mM to about 10 mM L-methionine, or a pharmaceutically acceptable salt thereof;
e) about 6% to about 8% w/v sucrose; and
f) about 0.01% to about 0.04% w/v polysorbate 80.

In one embodiment of the foregoing aspects of the invention, the formulation has a pH between about 5.0 and about 6.0. In one embodiment, the formulation has a pH between 5.3 and 5.8. In one embodiment, the formulation has a pH around 5.5.

In one embodiment of the foregoing aspects of the invention, the buffer is a histidine buffer. In another embodiment, the histidine buffer is present at a concentration of about 5 mM to about 20 mM. In one embodiment of the foregoing aspects of the invention, the histidine buffer is present at a concentration of about 8 mM to about 12 mM. In one embodiment of the foregoing aspects of the invention, the histidine buffer is L-histidine. In one embodiment of the foregoing aspects of the invention, the buffer is about 10 mM histidine. In one embodiment of the foregoing aspects of the invention, the buffer is about 10 mM L-histidine.

In one embodiment of the foregoing aspects of the invention, the anti-oxidant is L-methionine or a pharmaceutically acceptable salt thereof. In one embodiment of the foregoing aspects of the invention, the anti-oxidant is about 1 mM to about 30 mM L-methionine or a pharmaceutically acceptable salt thereof. In one embodiment of the foregoing aspects of the invention, the anti-oxidant is about 1 mM to about 20 mM L-methionine or a pharmaceutically acceptable salt thereof. In a further embodiment, the anti-oxidant is L-methionine or a pharmaceutically acceptable salt thereof, which is present at a concentration of about 5 mM to about 15 mM. In another embodiment, the L-methionine, or a pharmaceutically acceptable salt thereof is present at a concentration of about 10 mM. In one embodiment of the foregoing aspects of the invention, the L-methionine or a pharmaceutically acceptable salt thereof is L-methionine-HCl.

In one embodiment of the foregoing aspects of the invention, the non-reducing dissacharide is sucrose or trehalose. In one embodiment, the sucrose, or trehalose is about 3% to about 10% weight/volume (w/v). In another embodiment, the sucrose, or trehalose is about 6% to about 8% weight/volume (w/v). In one embodiment, the sucrose is present at approximately 7% w/v.

In a further embodiment of the foregoing aspects of the invention, the non-ionic surfactant is polysorbate 80, 60, 40 or 20. In another embodiment, the non-ionic surfactant is present at approximately 0.005-0.10% w/v. In another embodiment, the non-ionic surfactant is present at approximately 0.005-0.02% w/v. In another embodiment, the non-ionic surfactant is polysorbate 80, which is present at approximately 0.02% w/v. In one embodiment of the foregoing aspects of the invention, the non-ionic surfactant is about 0.01% to about 0.04% w/v polysorbate 80. In one embodiment of the foregoing aspects of the invention, the non-ionic surfactant is about 0.02% w/v polysorbate 80.

### Anti-PD-1 Antibodies and Antigen-Binding Fragments Thereof

The invention provides stable biological formulations comprising antibodies or antigen binding fragments thereof, which specifically bind to human PD-1 (e.g. a human or humanized anti-PD-1 antibody) and a PH20 variant or fragments thereof, as well as methods for using the formulations of the invention. In particular embodiments, the anti-PD-1 antibody is selected from pembrolizumab and nivolumab. In specific embodiments, the anti-PD-1 antibody is pembrolizumab or pembrolizumab variant. In alternative embodiments, the anti-PD-1 antibody is nivolumab. Table 2 provides amino acid sequences for exemplary anti-human PD-1 antibodies pembrolizumab and nivolumab.

In some embodiments, an anti-human PD-1 antibody or antigen binding fragment thereof for use in the formulations of the invention comprises a light chain variable region comprising three light chain CDRs of CDRL1, CDRL2 and CDRL3 and a heavy chain variable region comprising three heavy chain CDRs of CDRH1, CDRH2 and CDRH3.

In one embodiment of the invention, CDRL1 is SEQ ID NO:1 or a variant of SEQ ID NO:1, CDRL2 is SEQ ID NO:2 or a variant of SEQ ID NO:2, and CDRL3 is SEQ ID NO:3 or a variant of SEQ ID NO:3.

In one embodiment, CDRH1 is SEQ ID NO:6 or a variant of SEQ ID NO:6, CDRH2 is SEQ ID NO: 7 or a variant of SEQ ID NO:7, and CDRH3 is SEQ ID NO:8 or a variant of SEQ ID NO:8.

In one embodiment, the three light chain CDRs are SEQ ID NO: 1, SEQ ID NO:2, and SEQ ID NO:3 and the three heavy chain CDRs are SEQ ID NO:6, SEQ ID NO:7 and SEQ ID NO:8.

In an alternative embodiment of the invention, CDRL1 is SEQ ID NO:11 or a variant of SEQ ID NO:11, CDRL2 is SEQ ID NO:12 or a variant of SEQ ID NO:12, and CDRL3 is SEQ ID NO:13 or a variant of SEQ ID NO: 13.

In one embodiment, CDRH1 is SEQ ID NO: 16 or a variant of SEQ ID NO:16, CDRH2 is SEQ ID NO:17 or a variant of SEQ ID NO:17, and CDRH3 is SEQ ID NO:18 or a variant of SEQ ID NO:18.

In an alternative embodiment, the three light chain CDRs are SEQ ID NO:11, SEQ ID NO:12, and SEQ ID NO:13 and the three heavy chain CDRs are SEQ ID NO:16, SEQ ID NO:17 and SEQ ID NO:18.

Anti-PD-1 binding fragments of the formulations of the invention comprise a light chain variable region and a heavy chain variable region. In some embodiments, the light chain variable region comprises SEQ ID NO:4 or a variant of SEQ ID NO:4, and the heavy chain variable region comprises SEQ ID NO:9 or a variant of SEQ ID NO:9. In further embodiments, the light chain variable region comprises SEQ ID NO:14 or a variant of SEQ ID NO:14, and the heavy chain variable region comprises SEQ ID NO:19 or a variant of SEQ ID NO:19. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the formulations of the invention, the antibody or antigen binding fragment comprises a light chain variable region comprising or consisting of SEQ ID NO:4 and a heavy chain variable region comprising or consisting SEQ ID NO:9. In a further embodiment, the antibody or antigen binding fragment comprises a light chain variable region comprising or consisting of SEQ ID NO:14 and a heavy chain variable region comprising or consisting of SEQ ID NO:19.

In another embodiment, the formulations of the invention comprise an antibody or antigen binding fragment that has a V_{L} domain and/or a V_{H} domain with at least 95%, 90%, 85%, 80%, 75% sequence homology to one of the V_{L} domains or V_{H} domains described above, and exhibits specific binding to PD-1. In another embodiment, the antibody or antigen binding fragment of the formulations of the invention comprises V_{L} and V_{H} domains having up to 1, 2, 3, 4, or 5 or more amino acid substitutions, and exhibits specific binding to PD-1.

In any of the embodiments above, the anti-PD-1 antibody may be a full-length anti-PD-1 antibody that specifically binds human PD-1. In certain embodiments, the full-length anti-PD-1 antibody selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the invention were to call for altered effector functions, a heavy chain constant domain other than IgG1 may be used. Although IgG1 antibodies provide for long half-life and effector functions, such as complement activation and antibody-dependent cellular cytotoxicity, such activities may not be desirable for all uses of the antibody. In such instances an IgG4 constant domain, for example, may be used.

In embodiments of the invention, the anti-PD-1 antibody comprises a light chain comprising or consisting of a sequence of amino acid residues as set forth in SEQ ID NO:5 and a heavy chain comprising or consisting of a sequence of amino acid residues as set forth in SEQ ID NO:10. In alternative embodiments, the anti-PD-1 antibody comprises a light chain comprising or consisting of a sequence of amino acid residues as set forth in SEQ ID NO:15 and a heavy chain comprising or consisting of a sequence of amino acid residues as set forth in SEQ ID NO:20. In some formulations of the invention, the anti-PD-1 antibody is pembrolizumab, a pembrolizumab biosimilar or pembrolizumab variant. In some formulations of the invention, the anti-PD-1 antibody is nivolumab or a nivolumab biosimilar.

Ordinarily, amino acid sequence variants of the anti-PD-1 antibodies and antigen binding fragments of the invention will have an amino acid sequence having at least 75% amino acid sequence identity with the amino acid sequence of a reference antibody or antigen binding fragment (e.g. heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

Additional anti-PD-1 antibodies contemplated for use herein include MEDI0680 (U.S. Pat. No. 8,609,089), BGB-A317 (U.S. Patent publ. no. 2015/0079109), INCSHR1210 (SHR-1210) (PCT International application publ. no. WO2015/085847), REGN-2810 (PCT International application publ. no. WO2015/112800), PDR001 (PCT International application publ. no. WO2015/112900), TSR-042 (ANB011) (PCT International application publ. no. WO2014/179664) and STI-1110 (PCT International application publ. no. WO2014/194302); the humanized antibodies h409A11, h409A16 and h409A17, which are described in WO2008/156712, and AMP-514, which is being developed by MedImmune (publications here incorporated herein by reference in its entirety); cemiplimab; camrelizumab; sintilimab; tislelizumab; and toripalimab.

TABLE 2
Exemplary PD-1 Antibody Sequences

| | | |
|---|---|---|
| Antibody | Amino Acid Sequence | SEQ ID NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable Region | EIVLTQSPATLSLSPGERATLSCRASKGVSTSGYSYLHWY | 4 |
| | QQKPGQAPRLLIYLASYLESGVPARFSGSGSGTDFTLTIS | |
| | SLEPEDFAVYYCQHSRDLPLTFGGGTKVEIK | |
| Light Chain | EIVLTQSPATLSLSPGERATLSCRASKGVSTSGYSYLHWY | 5 |
| | QQKPGQAPRLLIYLASYLESGVPARFSGSGSGTDFTLTIS | |
| | SLEPEDFAVYYCQHSRDLPLTFGGGTKVEIKRTVAAPSVF | |
| | IFPPSDEQLKSGTASVVCLLNNFYPREAKVQWKVDNALQS | |
| | GNSQESVTEQDSKDSTYSLSSTLTLSKADYEKHKVYACEV | |
| | THQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable Region | QVQLVQSGVEVKKPGASVKVSCKASGYTFTNYYMYWVRQA | 9 |
| | PGQGLEWMGGINPSNGGTNFNEKFKNRVTLTTDSSTTTAY | |
| | MELKSLQFDDTAVYYCARRDYRFDMGFDYWGQGTTVTVSS | |
| Heavy Chain | QVQLVQSGVEVKKPGASVKVSCKASGYTFTNYYMYWVRQA | 10 |
| | PGQGLEWMGGINPSNGGTNFNEKFKNRVTLTTDSSTTTAY | |
| | MELKSLQFDDTAVYYCARRDYRFDMGFDYWGQGTTVTVSS | |
| | ASTKGPSVFPLAPCSRSTSESTAALGCLVKDYFPEPVTVS | |
| | WNSGALTSGVHTFPAVLQSSGLYSLSSVVTVPSSSLGTKT | |
| | YTCNVDHKPSNTKVDKRVESKYGPPCPPCPAPEFLGGPSV | |
| | FLFPPKPKDTLMISRTPEVTCVVVDVSQEDPEVQFNWYVD | |
| | GVEVHNAKTKPREEQFNSTYRVVSVLTVLHQDWLNGKEYK | |
| | CKVSNKGLPSSIEKTISKAKGQPREPQVYTLPPSQEEMTK | |
| | NQVSLTCLVKGFYSPDIAVEWESNGQPENNYKTTPPVLDS | |
| | DGSFFLYSRLTVDKSRWQEGNVFSCSVMHEALHNHYTQKS | |
| | LSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable Region | EIVLTQSPATLSLSPGERATLSCRASQSVSSYLAWYQQKP | 14 |
| | GQAPRLLIYDASNRATGIPARFSGSGSGTDFTLTISSLEP | |
| | EDFAVYYCQQSSNWPRTFGQGTKVEIK | |
| Light Chain | EIVLTQSPATLSLSPGERATLSVRASQSVSSYLAWYQQKP | 15 |
| | GQAPRLLIYDASNRATGIPARFSGSGSGTDFTLTISSLEP | |
| | EDFAVYYCQQSSNWPRTFGQGTKVEIKRTVAAPSVFIFPP | |
| | SDEQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable Region | QVQLVESGGGVVQPGRSLRLDCKASGITFSNSGMHWVRQA | 19 |
| | PGKGLEWVAVIWYDGSKRYYADSVKGRFTISRDNSKNTLF | |
| | LQMNSLRAEDTAVYYCATNDDYWGQGTLVTVSS | |
| Heavy Chain | QVQLVESGGGVVQPGRSLRLDCKASGITFSNSGMHWVRQA | 20 |
| | PGKGLEWVAVIWYDGSKRYYADSVKGRFTISRDNSKNTLF | |
| | LQMNSLRAEDTAVYYCATNDDYWGQGTLVTVSSASTKGPS | |
| | VFPLAPCSRSTSESTAALGCLVKDYFPEPVTVSWNSGALT | |
| | SGVHTFPAVLQSSGLYSLSSVVTVPSSSLGTKTYTCNVDH | |
| | KPSNTKVDKRVESKYGPPCPPCPAPEFLGGPSVFLFPPKP | |
| | KDTLMISRTPEVTCVVVDVSQEDPEVQFNWYVDGVEVHNA | |
| | KTKPREEQFNSTYRVVSVLTVLHQDWLNGKEYKCKVSNKG | |
| | LPSSIEKTISKAKGQPREPQVYTLPPSQEEMTKNQVSLTC | |
| | LVKGFYPSDIAVEWESNGQPENNYKTTPPVLDSDGSFFLY | |
| | SRLTVDKSRWQEGNVFSCSVMHEALHNHYTQKSLSLSLGK | |

In some embodiments of the formulations of the invention, the anti-PD-1 antibody or antigen binding fragment thereof (e.g. pembrolizumab) is present in a concentration of about 20 mg/mL to about 200 mg/mL. In some embodiments of the formulations of the invention, the anti-PD-1 antibody or antigen binding fragment thereof (e.g. pembrolizumab) is present in a concentration of about 90 mg/mL to about 200 mg/mL. In alternative embodiments, the anti-PD-1 antibody or antigen binding fragment thereof (e.g. pembrolizumab) is present in a concentration of about 100 mg/ml to about 185 mg/ml. In alternative embodiments, the anti-PD-1 antibody or antigen binding fragment thereof (e.g. pembrolizumab) is present in a concentration of about 25 mg/mL, about 50 mg/mL, about 75 mg/mL, about 90 mg/mL, about 100 mg/mL, about 120 mg/ml, about 125 mg/mL, about 130 mg/mL, about 150 mg/mL, about 165 mg/mL, about 167 mg/mL, about 185 mg/mL, and about 200 mg/mL.

In one embodiment, the anti-PD-1 antibody or antigen binding fragment thereof (e.g. pembrolizumab) is present in a concentration of about 165 to about 170 mg/mL.

In one embodiment, the anti-PD-1 antibody or antigen binding fragment thereof (e.g. pembrolizumab) is present in a concentration of about 165 mg/mL.

In one embodiment, the anti-PD-1 antibody or antigen binding fragment thereof (e.g. pembrolizumab) is present in a concentration of about 130 mg/mL.

In one embodiment, the anti-PD-1 antibody or antigen binding fragment thereof (e.g. pembrolizumab) is present in a concentration of about 120 mg/mL.

In one embodiment, the anti-PD-1 antibody or antigen binding fragment thereof (e.g. pembrolizumab) is present in a concentration of about 100 mg/mL.

In additional embodiments, the anti-PD-1 antibody or antigen binding fragment thereof (e.g. pembrolizumab) is present in a concentration of from about 75 mg/mL to about 200 mg/mL; from about 100 mg/mL to about 200 mg/mL; from about 25 mg/mL to about 175 mg/mL; from about 50 mg/mL to about 175 mg/mL; from about 75 mg/mL to about 175 mg/mL; from about 100 mg/mL to about 175 mg/mL; from about 25 mg/mL to about 150 mg/mL; from about 50 mg/mL to about 150 mg/mL; from about 75 mg/mL to about 150 mg/mL; from about 100 mg/mL to about 150 mg/mL; from about 25 mg/mL to about 125 mg/mL; from about 50 mg/mL to about 125 mg/mL; from about 75 mg/mL to about 125 mg/mL; from about 25 mg/mL to about 100 mg/mL, from about 125 mg/mL to about 175 mg/mL, from about 125 mg/mL to about 200 mg/mL, or from about 25 mg/mL to 200 mg/mL.

### PH20 Variants and Fragments Thereof

In one embodiment, the PH20 variant or fragment thereof further comprises an amino acid residue substitution at one or more positions selected from the group consisting of T341, L342, S343, 1344, and N363. In one embodiment, the PH20 variant or fragment thereof further comprises one or more amino acid residue substitutions selected from the group consisting of T341A, T341C, T341D, T341G, T341S, L342W, S343E, I344N and N363G.

In one embodiment of the PH20 variant or fragment thereof, the amino acid residue substitutions are selected from the following amino acid residue substitution groups:
(a) T341S, L342W, S343E, I344N, M345T, S347T, M348K, K349E, L352Q, L353A, L354I, D355K, N356E, E359D and I361T;
(b) L342W, S343E, I344N, M345T, S347T, M348K, K349E, L352Q, L353A, L354I, D355K, N356E, E359D and I361T;
(c) M345T, S347T, M348K, K349E, L352Q, L353A, L354I, D355K, N356E, E359D, 1361T and N363G;
(d) T341G, L342W, S343E, I344N, M345T, S347T, M348K, K349E, L352Q, L353A, L354I, D355K, N356E, E359D and I361T;
(e) T341A, L342W, S343E, I344N, M345T, S347T, M348K, K349E, L352Q, L353A, L354I, D355K, N356E, E359D and I361T;
(f) T341C, L342W, S343E, I344N, M345T, S347T, M348K, K349E, L352Q, L353A, L354I, D355K, N356E, E359D and I361T;
(g) T341D, L342W, S343E, I344N, M345T, S347T, M348K, K349E, L352Q, L353A, L354I, D355K, N356E, E359D and I361T;
(h) I344N, M345T, S347T, M348K, K349E, L352Q, L353A, L354I, D355K, N356E, E359D and I361T; and
(i) S343E, I344N, M345T, S347T, M348K, K349E, L352Q, L353A, L354I, D355K, N356E, E359D and I361T.
   In one embodiment of the PH20 variant or fragment thereof, the amino acid residue substitutions consists of T341S, L342W, S343E, I344N, M345T, S347T, M348K, K349E, L352Q, L353A, L354I, D355K, N356E, E359D and I361T.

In one aspect of the foregoing embodiments of a PH20 variant fragment, the PH20 variant fragment has an N-terminus deletion of amino acid residues 1-36, 1-37, 1-38, 1-39, 1-40, 1-41, or 1-42 of SEQ ID NO: 21. In another embodiment, the PH20 variant fragment has an N-terminus deletion of amino acid residues 1-36 of SEQ ID NO: 21. In another embodiment, the PH20 variant fragment has an N-terminus deletion of amino acid residues 1-37 of SEQ ID NO: 21. In another embodiment, the PH20 variant fragment has an N-terminus deletion of amino acid residues 1-38 of SEQ ID NO: 21.

In another aspect of the foregoing embodiments of a PH20 variant fragment, the PH20 variant fragment has a C-terminus deletion of amino acid residue(s) 455-509, 456-509, 457-509, 458-509, 459-509, 460-509, 461-509, 462-509, 463-509, 464-509, 465-509, 466-509, 467-509, 468-509, 469-509, 470-509, 471-509, 472-509, 473-509, 474-509, 475-509, 476-509, 477-509, 478-509, 479-509, 480-509, 481-509, 482-509, 483-509, 484-509, 485-509, 486-509, 487-509, 488-509, 489-509, 490-509, 491-509, 492-509, 493-509, 494-509, 495-509, 496-509, 497-509, 498-509, 499-509, 500-509, 501-509, 502-509, 503-509, 504-509, 505-509, 506-509, 507-509, 508-509, or 509, wherein the numbering is in reference to SEQ ID NO: 21. In one embodiment, the PH20 variant fragment thereof has a C-terminus deletion of amino acid residues 455-509, 458-509, 461-509, 464-509, 465-509, 466-509, 467-509, 468-509, 470-509, 471-509, 472-509, 473-509, 474-509, 475-509, 476-509, 478-509, 480-509, 482-509, 484-509, 486-509, 488-509, or 490-509, wherein the numbering is in reference to SEQ ID NO: 21. In one embodiment, the PH20 variant fragment has a C-terminus deletion of amino acid residues 468-509, wherein the numbering is in reference to SEQ ID NO: 21.

In one embodiment, the PH20 variant fragment consists of the amino acid sequence set forth in SEQ ID NO: 22 or 23. In other embodiments, the PH20 variant or fragment thereof is any of the sequences disclosed in Table 11 of EP3636752.

TABLE 3
Hyaluronidase and exemplary variants

| Protein | Sequence | SEQ ID NO: |
|---|---|---|
| Wild-type | MGVLKFKHIFFRSFVKSSGVSQIVFTFLLIPCCLTLNFRAPPVIPNVPFL | 21 |
| PH20 | WAWNAPSEFCLGKFDEPLDMSFSFIGSPRINATGQGVTIFYVDRLGYYPY | |
| | IDSITGVTVNGGIPQKISLQDHLDKAKKDITFYMPVDNLGMAVIDWEEWR | |
| | PTWARNWKPKDVYKNRSIELVQQQNVQLSLTEATEKAKQEFEKAGKDFLV | |
| | ETIKLGKLLRPNHLWGYYLFPDCYNHHYKKPGYNGSCFNVEIKRNDDLSW | |
| | LWNESTALYPSIYLNTQQSPVAATLYVRNRVREAIRVSKIPDAKSPLPVF | |
| | AYTRIVFTDQVLKFLSQDELVYTFGETVALGASGIVIWGTLSIMRSMKSC | |
| | LLLDNYMETILNPYIINVTLAAKMCSQVLCQEQGVCIRKNWNSSDYLHLN | |
| | PDNFAIQLEKGGKFTVRGKPTLEDLEQFSEKFYCSCYSTLSCEKEADVKD | |
| | TDAVDVCIADGVCIDAFLKPPMETEEPQIFYNASPSTLSATMFIVSILFL | |
| | IISSVASL | |
| PH20 | LNFRAPPVIPNVPFLWAWNAPSEFCLGKFDEPLDMSLFSFIGSPRINATG | 22 |
| variant | QGVTIFYVDRLGYYPYIDSITGVTVNGGIPQKISLQDHLDKAKKDITFYM | |
| fragment 1 | PVDNLGMAVIDWEEWRPTWARNWKPKDVYKNRSIELVQQQNVQLSLTEAT | |
| | EKAKQEFEKAGKDFLVETIKLGKLLRPNHLWGYYLFPDCYNHHYKKPGYN | |
| | GSVFNVEIKRNDDLSWLWNESTALYPSIYLNTQQSPVAATLYVRNRVREA | |
| | IRVSKIPDAKSPLPVFAYTRIVFTDQVLKFLSQDELVYTFGETVALGASG | |
| | IVIWGTLSITRTKESCQAIKEYMDTTLNPYIINVTLAAKMCSQVLCQEQG | |
| | VCIRKNWNSSDYLHLNPDNFAIQLEKGGKFTVRGKPTLEDLEQFSEKFYC | |
| | SCYSTLSCKEKADVKDTDAVDVCIADGVCIDAFLKPPMETEEPQIFYNAS | |
| | PSTLS | |
| PH20 | FRAPPVIPNVPFLWAWNAPSEFVLGKFDEPLDMSLFSFIGSPRINATCQG | 23 |
| variant | VTIFYVDRLGYYPYIDSITGVTVNGGIPQKISLQDHLDKAKKDITFYMPV | |
| fragment 2 | DNLGMAVIDWEEWRPTWARNWKPKDVYKNRSIELVQQQNVQLSLTEATEK | |
| | AKQEFEKAGKDFLVETIKLGKLLRPNHLWGYYLFPDCYNHHYKKPGYNGS | |
| | CFNVEIKRNDDLSWLWNESTALYPSIYLNTQQSPVAATLYVRNRVREAIR | |
| | SKIPDAKSPLPVFAYTRIVFTDQVLKFLSQDELVYTFGETVALGASGIVI | |
| | WGSWENTRTKESCQAIKEYMDTTLNPYIINVTLAAKMCSQVLCQEQGVCI | |
| | RKNWNSSDYLHLNPDNFAIQLEKGGKFTVRGKPTLEDLEQFSEKFYCSCY | |
| | STLSCKEKADVKDTDAVDVCIADGVCIDAF | |

In some embodiments of the formulations of the invention, the PH20 variant or fragment thereof is present in a concentration of about 0.006 mg/mL. In another embodiment, the concentration of the PH20 variant or fragment thereof is about 0.009 mg/mL. In another embodiment, the concentration of the PH20 variant or fragment thereof is about 0.012 mg/mL. In another embodiment, the concentration of the PH20 variant or fragment thereof is about 0.018 mg/mL. In another embodiment, the concentration of the PH20 variant or fragment thereof is about 0.024 mg/mL. In another embodiment, the concentration of the PH20 variant or fragment thereof is about 0.030 mg/mL. In another embodiment, the concentration of the PH20 variant or fragment thereof is about 0.036 mg/mL. In a further embodiment, the concentration of the PH20 variant or fragment thereof is about 0.006-0.036 mg/mL. In a further embodiment, the concentration of the PH20 variant or fragment thereof is about 0.012-0.030 mg/mL. In a further embodiment, the concentration of the PH20 variant or fragment thereof is about 0.006-0.030 mg/mL. In a further embodiment, the concentration of the PH20 variant or fragment thereof is about 0.006-0.012 mg/mL.

In some embodiments of the formulations of the invention, the PH20 variant or fragment thereof is present in a concentration of about 1000 U/ml. In another embodiment, the concentration of the PH20 variant or fragment thereof is about 1500 U/ml. In another embodiment, the concentration of the PH20 variant or fragment thereof is about 2000 U/ml. In another embodiment, the concentration of the PH20 variant or fragment thereof is about 3000 U/ml. In another embodiment, the concentration of the PH20 variant or fragment thereof is about 4000 U/ml. In another embodiment, the concentration of the PH20 variant or fragment thereof is about 5000 U/ml. In another embodiment, the concentration of the PH20 variant or fragment thereof is about 6000 U/ml. In a further embodiment, the concentration of the PH20 variant or fragment thereof is about 1000-6000 U/ml. In a further embodiment, the concentration of the PH20 variant or fragment thereof is about 2000-5000 U/ml.

In some embodiments of the formulations of the invention, the PH20 variant or fragment thereof is present in a concentration of about 0.0009 mg/mL. In another embodiment, the concentration of the PH20 variant or fragment thereof is about 0.0018 mg/mL. In another embodiment, the concentration of the PH20 variant or fragment thereof is about 0.0036 mg/mL. In another embodiment, the concentration of the PH20 variant or fragment thereof is about 0.0045 mg/mL. In a further embodiment, the concentration of the PH20 variant or fragment thereof is about 0.0009-0.030 mg/mL.

In some embodiments of the formulations of the invention, the PH20 variant or fragment thereof is present in a concentration of about 150 U/ml. In another embodiment, the concentration of the PH20 variant or fragment thereof is about 300 U/ml. In another embodiment, the concentration of the PH20 variant or fragment thereof is about 600 U/ml. In another embodiment, the concentration of the PH20 variant or fragment thereof is about 750 U/ml. In a further embodiment, the concentration of the PH20 variant or fragment thereof is about 150-5000 U/ml.

### Formulation Excipients

In embodiments of the invention, the non-reducing dissacharide is sucrose. In additional embodiments, the non-reducing dissacharide is trehalose.

In some embodiments, the non-reducing dissacharide is about 6% to about 8% w/v sucrose. In some embodiments, the non-reducing dissacharide is about 6% to about 8% w/v trehalose.

In still further embodiments, the sucrose, trehalose is present in an amount of about 6% w/v, about 6.25% w/v, about 6.5% w/v, about 6.75% w/v, about 7% w/v, about 7.25% w/v, about 7.5% w/v, about 7.75% w/v or about 8% w/v.

In addition to an anti-PD-1 antibody or antigen binding fragment thereof and PH20 variant or fragment thereof, and a non-reducing dissacharide in the amounts/concentrations specified above, the formulations of the invention may also comprise a buffer. In some embodiments the buffer is present in an amount of about 5 mM to about 20 mM. In further embodiments, the buffer has a pH in a range of about 5.0 to about 6.0. In still further embodiments, the pH is from about 5.3 to about 5.8. In other embodiments, the pH is from about 6.0 to about 6.4.

In particular embodiments, the buffer has a pH of about 5.0, about 5.1, about 5.2, about 5.3, about 5.4, about 5.5, about 5.6, about 5.7, about 5.8, about 5.9, about 6.0, about 6.2 or about 6.4. In specific embodiments of the invention, the buffer is histidine or acetate at a pH of about 5.0 to about 6.0. In some embodiments, the buffer is an L-histidine buffer. In embodiments where the formulation is lyophilized, it is preferred that the buffer is not acetate because acetate buffer systems are not compatible with the lyophilization process.

When a range of pH values is recited, such as “a pH between pH 5.5 and 6.0,” the range is intended to be inclusive of the recited values. Unless otherwise indicated, for a lyophilized formulation, the pH refers to the pH after reconstitution of the lyophilized formulations of the invention. The pH is typically measured at 25 °C using standard glass bulb 30 pH meter. As used herein, a solution comprising “histidine buffer at pH X” refers to a solution at pH X and comprising the histidine buffer, i.e. the pH is intended to refer to the pH of the solution.

In addition to an anti-PD-1 antibody or antigen binding fragment thereof and PH20 variant or fragment thereof, a non-reducing dissacharide, and a buffer in the amounts/concentrations specified above, the formulations of the invention may also comprise an anti-oxidant. In embodiments of the invention, the anti-oxidant is methionine. In embodiments of the invention, the anti-oxidant is L-methionine, or a pharmaceutically acceptable salt thereof. In further embodiments, the methionine is L-methionine. In other embodiments, the anti-oxidants is L-methionine HCl.

In some embodiments, the anti-oxidant (e.g. L-methionine) is present in the formulations of the invention in an amount of 1 mM to about 20 mM. In another embodiment, the anti-oxidant is present in an amount of about 5 mM to about 20 mM. In a further embodiment, the anti-oxidant is present in about 5 mM to about 15 mM. In a further embodiment, the anti-oxidant is present in about 5 mM to about 10 mM. In additional embodiments, the anti-oxidant is present in an amount of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM.

In addition to an anti-PD-1 antibody or antigen binding fragment thereof, a PH20 variant or fragment thereof, a non-reducing dissacharide, a buffer, and an anti-oxidant in the amounts/concentrations specified above, the formulations of the invention may also comprise a surfactant. Surfactants that may be useful in the formulations of the invention include, but are not limited to: nonionic surfactants such as polyoxyethylene sorbitan fatty acid esters (Polysorbates, sold under the trade name Tween® (Uniquema Americas LLC, Wilmington, DE)) including Polysorbate-20 (polyoxyethylene sorbitan monolaurate), Polysorbate-40 (polyoxyethylene sorbitan monopalmitate), Polysorbate-60 (polyoxyethylene sorbitan monostearate), and Polysorbate-80 (polyoxyethylene sorbitan monooleate).

The amount of surfactant to be included in the formulations of the invention is an amount sufficient to perform the desired function, i.e. a minimal amount necessary to stabilize the active pharmaceutical ingredient (i.e. the anti-PD-1 antibody or antigen binding fragment thereof or PH20 variant or fragment thereof) in the formulation. Typically, the surfactant is present in a concentration of from about 0.005% to about 0.1% w/v. In some embodiments of this aspect of the invention, the surfactant is present in the formulation in an amount from about 0.01% to about 0.04%; from about 0.01% to about 0.03%, from about 0.01% to about 0.02%, from about 0.015% to about 0.04%; from about 0.015% to about 0.03%, from about 0.015% to about 0.02%, from about 0.02% to about 0.04%, from about 0.02% to about 0.035%, or from about 0.02% to about 0.03%. In specific embodiments, the surfactant is present in an amount of about 0.02%. In alternative embodiments, the surfactant is present in an amount of about 0.01%, about 0.015%, about 0.025%, about 0.03%, about 0.035%, or about 0.04%.

In exemplary embodiments of the invention, the surfactant is a nonionic surfactant selected from the group consisting of. Polysorbate 20, and Polysorbate 80. In preferred embodiments, the surfactant is Polysorbate 80.

In specific embodiments, the formulations of the invention comprise about 0.01% to about 0.04% PS80. In further embodiments, the formulations of the invention comprise PS80 in an amount of about 0.008%, about 0.01%, about 0.015%, about 0.02%, about 0.025%, about 0.03%, about 0.035%, about 0.04% or about 0.045%. In particular embodiments, the formulations of the invention comprise about 0.02% PS80.

The invention also provides a formulation as described herein, wherein the formulation is contained in a glass vial or injection device (e.g. a syringe). In one aspect, the formulation is for subcutaneous administration. In one embodiment, the viscosity of the formulation is in the range of 7-90 cP at 5 °C. In another embodiment, the viscosity of the formulation is in the range of 7-30 cP at 5 °C. In another embodiment, the viscosity of the formulation is in the range of 7-50 cP at 20 °C. In a further embodiment, the viscosity of the formulation is in the range of 7-20 cP at 20 °C. In one embodiment, the viscosity is measured using the USP <913> technique with a MiniVisII viscometer by Grabner Instruments.

In additional embodiments, the invention provides formulations as described herein, wherein after storage of the formulation at 2-8 °C for 3 or 6 months, the % high molecular weight species as measured by HP-SEC is ≤1 or 0.5%.

In additional embodiments, the invention provides formulations as described herein, wherein after storage of the formulation at 25 °C for 6, 3 or 1 month, the % high molecular weight species as measured by HP-SEC is ≤2%.

In additional embodiments, the invention provides formulations as described herein, wherein after storage of the formulation at 40 °C for 3 months, the % high molecular weight species as measured by HP-SEC is ≤4%, or ≤5.0%.

In additional embodiments, the invention provides formulations as described herein, wherein after storage of the formulation at 40 °C for 6 months, the % high molecular weight species as measured by HP-SEC is ≤11%, or ≤12.0%.

In further embodiments, the invention provides formulations as described herein, wherein after storage of the formulation at 5 °C for 1, 3 or 6 months, the % monomer as measured by HP-SEC is ≥99.5%.

In further embodiments, the invention provides formulations as described herein, wherein after storage of the formulation at 25 °C for 1, 3 or 6 months, the % monomer as measured by HP-SEC is ≥98%.

In further embodiments, the invention provides formulations as described herein, wherein after storage of the formulation at 40 °C for 3 months, the % monomer as measured by HP-SEC is ≥95%.

In further embodiments, the invention provides formulations as described herein, wherein after storage of the formulation at 5 °C for 1, 3 or 6 months, the % acid variants as measured by IEX is ≤20%.

In further embodiments, the invention provides formulations as described herein, wherein after storage of the formulation at 25 °C for 3 months, the % acid variants as measured by IEX is ≤24 or 25%.

In further embodiments, the invention provides formulations as described herein, wherein after storage of the formulation at 40 °C for 3 months, the % acid variants as measured by IEX is ≤555%.

### Specific Aspects and Embodiments of the Invention

In one embodiment, the invention provides a formulation, comprising:
a) about 100 to about 165 mg/mL of an anti-human PD-1 antibody, or antigen binding fragment thereof,
b) about 0.012-0.030 mg/ml of PH20 variant or fragment thereof,
c) about 10 mM histidine buffer;
d) optionally, about 10 mM L-methionine, or a pharmaceutically acceptable salt thereof,
e) about 7% w/v sucrose; and
f) about 0.02% w/v polysorbate 80.

In one embodiment, the invention provides a formulation, comprising:
a) about 130 mg/mL of an anti-human PD-1 antibody, or antigen binding fragment thereof,
b) about 0.012 mg/ml of PH20 variant or fragment thereof,
c) about 10 mM histidine buffer;
d) optionally, about 10 mM L-methionine, or a pharmaceutically acceptable salt thereof,
e) about 7% w/v sucrose; and
f) about 0.02% w/v polysorbate 80.

In one embodiment, the invention provides a formulation, comprising:
a) about 130 mg/mL of an anti-human PD-1 antibody, or antigen binding fragment thereof,
b) about 0.024 mg/ml of PH20 variant or fragment thereof,
c) about 10 mM histidine buffer;
d) optionally, about 10 mM L-methionine, or a pharmaceutically acceptable salt thereof,
e) about 7% w/v sucrose; and
f) about 0.02% w/v polysorbate 80.

In another embodiment, the invention provides a formulation, comprising:
a) about 130 mg/mL of an anti-human PD-1 antibody, or antigen binding fragment thereof,
b) about 0.030 mg/ml of PH20 variant or fragment thereof,
c) about 10 mM histidine buffer;
d) optionally, about 10 mM L-methionine, or a pharmaceutically acceptable salt thereof,
e) about 7% w/v sucrose; and
f) about 0.02% w/v polysorbate 80.

In another embodiment, the invention provides a formulation, comprising:
a) about 165 mg/mL of an anti-human PD-1 antibody, or antigen binding fragment thereof,
b) about 0.012 mg/ml of PH20 variant or fragment thereof,
c) about 10 mM histidine buffer;
d) optionally, about 10 mM L-methionine, or a pharmaceutically acceptable salt thereof,
e) about 7% w/v sucrose; and
f) about 0.02% w/v polysorbate 80.

In another embodiment, the invention provides a formulation, comprising:
a) about 165 mg/mL of an anti-human PD-1 antibody, or antigen binding fragment thereof,
b) about 0.024 mg/ml of PH20 variant or fragment thereof,
c) about 10 mM histidine buffer;
d) optionally, about 10 mM L-methionine, or a pharmaceutically acceptable salt thereof,
e) about 7% w/v sucrose; and
f) about 0.02% w/v polysorbate 80.

In another embodiment, the invention provides a formulation, comprising:
a) about 165 mg/mL of an anti-human PD-1 antibody, or antigen binding fragment thereof,
b) about 0.030 mg/ml of PH20 variant or fragment thereof,
c) about 10 mM histidine buffer;
d) optionally, about 10 mM L-methionine, or a pharmaceutically acceptable salt thereof,
e) about 7% w/v sucrose; and
f) about 0.02% w/v polysorbate 80.

In any of the specific aspects and embodiments described herein, any anti-PD-1 antibody or antigen binding fragment thereof (i.e. an antibody or antigen binding fragment that specifically binds human PD-1, e.g. pembrolizumab or an antigen-binding fragment thereof) can be used. In particular embodiments, one of the anti-PD-1 antibodies, or antigen binding fragments thereof, described herein, e.g. described in the section entitled “Anti-PD-1 Antibodies and Antigen-Binding Fragments Thereof” is used.

In any of the specific aspects and embodiments described herein, any PH20 variant or fragment thereof can be used. In particular embodiments, one of PH20 variant or fragment thereof, e.g. described in the section entitled “PH20 variant or fragment thereof”, is used.

In some embodiments of the invention, any of the formulations described herein is in aqueous solution. In alternative embodiment, the invention provides lyophilized formulations made by lyophilizing an aqueous formulation to provide a reconstituted formulation of the invention, as discussed more fully, infra.

### Lyophilized Pharmaceutical Compositions

Lyophilized formulations of therapeutic proteins provide several advantages. Lyophilized formulations in general offer better chemical stability than solution formulations, and thus increased shelf-life. A lyophilized formulation may also be reconstituted at different concentrations depending on clinical factors, such as route of administration or dosing. For example, a lyophilized formulation may be reconstituted at a high concentration (i.e. in a small volume) if necessary for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically, the lyophilized formulation is prepared in anticipation of reconstitution at high concentration of drug product (DP), i.e. in anticipation of reconstitution in a low volume of water. Subsequent dilution with water or isotonic buffer can then readily be used to dilute the DP to a lower concentration. Typically, excipients are included in a lyophilized formulation of the invention at levels that will result in a roughly isotonic formulation when reconstituted at high DP concentration, e.g. for subcutaneous administration. Reconstitution in a larger volume of water to give a lower DP concentration will necessarily reduce the tonicity of the reconstituted solution, but such reduction may be of little significance in non-subcutaneous, e.g. intravenous, administration. If isotonicity is desired at lower DP concentration, the lyophilized powder may be reconstituted in the standard low volume of water and then further diluted with isotonic diluent, such as 0.9% sodium chloride.

In an embodiment of the invention, a formulation comprising the humanized anti-PD-1 antibody (or antigen binding fragment thereof) and PH20 variant or fragment thereof is formulated as a lyophilized powder for reconstituting and utilizing for subcutaneous administration. In certain embodiments, the lyophilized formulation is reconstituted with sterile water for injection prior to use. If desired, the reconstituted solution may be aseptically diluted with 0.9% sodium chloride Injection USP in a sterile IV container. In some embodiments, the target pH of the reconstituted formulation is 5.5±0.5. In various embodiments, the lyophilized formulation of the invention enables reconstitution of the anti-PD-1 antibody to high concentrations, such as about 20, 25, 30, 40, 50, 60, 75, 100, 125, 130, 150, 165, 175, 185 or 200 mg/mL.

Lyophilized formulations are by definition essentially dry, and thus the concept of concentration is not useful in describing them. Describing a lyophilized formulation in the terms of the weight of the components in a unit dose vial is more useful, but is problematic because it varies for different doses or vial sizes. In describing the lyophilized formulations of the invention, it is useful to express the amount of a component as the ratio of the weight of the component compared to the weight of the drug substance (DS) in the same sample (e.g. a vial). This ratio may be expressed as a percentage. Such ratios reflect an intrinsic property of the lyophilized formulations of the invention, independent of vial size, dosing, and reconstitution protocol.

In other embodiments, the lyophilized formulation of anti-human PD-1 antibody, or antigen binding fragment and PH20 variant or fragment thereof, is defined in terms of the pre-lyophilization solution used to make the lyophilized formulation, such as the pre-lyophilization solution. In one embodiment the pre-lyophilization solution comprises antibody, or antigen-binding fragment thereof, at a concentration of about 25-200 mg/mL and 0.0009-0.050 mg/ml of PH20 variant or fragment thereof. Such pre-lyophilization solutions may be at pH 4.4-6.0, e.g. preferably about pH 5.0-6.0, or about pH 5.5.

In yet other embodiments, the lyophilized formulation of anti-human PD-1 antibody, or antigen binding fragment and PH20 variant or fragment thereof, is defined in terms of the reconstituted solution generated from the lyophilized formulation. The invention provides a liquid formulation that is reconstituted from a lyophilized formulation. Reconstituted solutions may comprise antibody, or antigen-binding fragment thereof, at concentrations of about 25, 30, 40, 50, 60, 75, 80, 90, 100, 120, 130, 150, 165, 167, 185 or 200 mg/mL, and 0.0009-0.050 mg/ml (150, 300, 600, 900, 1000, 1500, 2000, 3000, 4000 or 5000 U/ml) of PH20 variant or fragment thereof. Such reconstituted solutions may be at about pH 5.5, or range from about pH 5.0 to about 6.0.

The lyophilized formulations of the invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −30 to 25 °C (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40 °C, depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

Reconstitution generally takes place at a temperature of about 25 °C to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution.

### Liquid Pharmaceutical Compositions

A liquid antibody formulation can be made by taking the drug substance (e.g., anti-human PD-1 antibody, and/or PH20 variant or fragment thereof) which is in liquid form (e.g., pembrolizumab or PH20 variant fragment 1 or 2 in an aqueous pharmaceutical formulation) and buffer exchanging it into the desired buffer. There is no lyophilization step in this embodiment. The drug substance in the final buffer is concentrated to a desired concentration. Excipients such as sucrose, methionine and polysorbate 80 are added to the drug substance and it is diluted using the appropriate buffer to final protein concentration. The final formulated drug substance is filtered, e.g. using 0.22 m filters, and filled into a final container (e.g. glass vials or syringes). Such a liquid formulation is exemplified by a final liquid formulation comprising 10 mM histidine pH 5.5, 7% sucrose, 0.02% polysorbate 80, 25-200 mg/mL pembrolizumab, and 0.0009-0.050 mg/ml of PH20 variant fragment 1 or 2.

### Methods of Use

The invention also relates to a method of treating cancer in a subject, the method comprising administering an effective amount of any of the formulations of the invention; i.e., any formulation described herein (including Specific Aspects and Embodiments of the Invention section herein), to the subject. In some embodiments of this method, the formulation is administered to the subject by subcutaneous administration.

In any of the methods of the invention, the cancer can be selected from the group consisting of: melanoma, lung cancer, head and neck cancer, bladder cancer, breast cancer, gastrointestinal cancer, multiple myeloma, hepatocellular cancer, merkel cell carcinoma, cutaneous squamous cell carcinoma, lymphoma, renal cancer, mesothelioma, ovarian cancer, esophageal cancer, anal cancer, biliary tract cancer, colorectal cancer, endometrial cancer, cervical cancer, thyroid cancer, salivary cancer, prostate cancer (e.g. hormone refractory prostate adenocarcinoma), pancreatic cancer, colon cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is mediastinal large B-cell lymphoma. In some embodiments, the lymphoma is diffuse large B-cell lymphoma (DLBCL).

In some embodiments, the breast cancer is triple negative breast cancer.

In further embodiments, the breast cancer is ER+/HER2-breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In some embodiments, the cancer is metastatic colorectal cancer with high levels of microsatellite instability (MSI-H).

In some embodiments, the cancer is a solid tumor with a high level of microsatellite instability (MSI-H).

In some embodiments, the cancer is a solid tumor with a high mutational burden.

In some embodiments, the cancer is selected from the group consisting of: melanoma, non-small cell lung cancer, relapsed or refractory classical Hodgkin lymphoma, head and neck squamous cell carcinoma, urothelial cancer, esophageal cancer, gastric cancer, DLBCL and hepatocellular cancer.

In other embodiments of the above treatment methods, the cancer is a Heme malignancy. In certain embodiments, the Heme malignancy is acute lymphoblastic leukemia (ALL), acute myeloid leukemia (AML), chronic lymphocytic leukemia (CLL), chronic myeloid leukemia (CMIL), DLBCL, EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin lymphoma (NHL), or small lymphocytic lymphoma (SLL).

Malignancies that demonstrate improved disease-free and overall survival in relation to the presence of tumor-infiltrating lymphocytes in biopsy or surgical material, e.g. melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

In some embodiments, the formulations of the invention are administered to a subject having a cancer characterized by elevated expression of PD-L1 and/or PD-L2 in tested tissue samples, including: ovarian, renal, colorectal, pancreatic, breast, liver, gastric, esophageal cancers and melanoma. Additional cancers that can benefit from treatment with anti-PD-1 antibodies such as humanized anti-PD-1 antibody pembrolizumab include those associated with persistent infection with viruses such as human immunodeficiency viruses, hepatitis viruses class A, B and C, Epstein Barr virus, human papilloma viruses that are known to be causally related to for instance Kaposi's sarcoma, liver cancer, nasopharyngeal cancer, lymphoma, cervical, vulval, anal, penile and oral cancers.

In one embodiment, the invention comprises a method of treating cancer in a human patient comprising administering any formulation of the invention to the patient.

In one embodiment, the invention comprises a method of treating unresectable or metastatic melanoma in a human patient comprising administering any formulation of the invention to the patient.

In one embodiment, the invention comprises a method of treating metastatic non-small cell lung cancer (NSCLC) in a human patient comprising administering a formulation of the invention to the patient. In specific embodiments, the patient has a tumor with high PD-L1 expression [(Tumor Proportion Score (TPS) 50%)] and was not previously treated with platinum-containing chemotherapy. In other embodiments, the patient has a tumor with PD-L1 expression (TPS≥1%) and was previously treated with platinum-containing chemotherapy. In still other embodiments, the patient has a tumor with PD-L1 expression (TPS≥1%) and was not previously treated with platinum-containing chemotherapy. In specific embodiments, the patient had disease progression on or after receiving platinum-containing chemotherapy.

In certain embodiments, the PD-L1 TPS is determined by an FDA-approved test.

In certain embodiments, the patient's tumor has no EGFR or ALK genomic aberrations.

In certain embodiments, the patient's tumor has an EGFR or ALK genomic aberration and had disease progression on or after receiving treatment for the EGFR or ALK aberration(s) prior to receiving the anti-PD-1 antibody, or antigen binding fragment thereof.

In one embodiment, the invention comprises a method of treating metastatic non-small cell lung cancer (NSCLC) in a human patient comprising: (1) administering a formulation of the invention to the patient, and (2) administering pemetrexed and carboplatin to the patient. In specific embodiments, the patient was not previously treated with an anti-cancer therapeutic prior to starting the combination treatment regimen with the formulation of the invention, pemetrexed and carboplatin.

In a certain embodiments, the patient has nonsquamous non-small cell lung cancer.

In certain embodiments, pemetrexed is administered to the patient in an amount of 500 mg/m². In sub-embodiments, pemetrexed is administered to the patient via intravenous infusion every 21 days. In specific embodiments, the infusion time is about 10 minutes.

In embodiments of the invention where the patient is treated with a formulation of the invention in combination with pemetrexed, the invention further comprises administering about 400 μg to about 1000 μg of folic acid to the patient once per day, beginning about 7 days prior to administering pemetrexed to the patient and continuing until about 21 days after the patient is administered the last dose of pemetrexed. In certain embodiments the folic acid is administered orally. In some embodiments, the invention further comprises administering about 1 mg of vitamin B₁₂ to the patient about 1 week prior to the first administration of pemetrexed and about every three cycles of pemetrexed administration (i.e., approximately every 9 weeks). In certain embodiments the vitamin B12 is administered intramuscularly. In certain embodiments, the invention further comprises administering about 4 mg of dexamethasone to the patient twice a day on the day before, the day of, and the day after pemetrexed administration. In certain embodiments the dexamethasone is administered orally.

In one embodiment, the invention comprises a method of treating recurrent or metastatic head and neck squamous cell cancer (HNSCC) in a human patient comprising administering any formulation of the invention to the patient. In certain embodiments, the patient was previously treated with platinum-containing chemotherapy. In certain embodiments, the patient had disease progression on or after platinum-containing chemotherapy. In specific embodiments, the patient's tumor expresses PD-L1 [Combined Positive Score (CPS)≥1].

In one embodiment, the invention comprises a method of treating refractory classical Hodgkin lymphoma (cHL) in a human patient comprising administering a formulation of the invention to the patient. In certain embodiments, the patient has relapsed after 3 or more lines of therapy for cHL. In specific embodiments, the patient is an adult patient. In alternative embodiments, the patient is a pediatric patient.

In one embodiment, the invention comprises a method of treating locally advanced or metastatic urothelial carcinoma in a human patient comprising administering a formulation of the invention to the patient. In certain embodiments, the patient is not eligible for cisplatin-containing chemotherapy. In certain embodiments, the patient has disease progression during or following platinum-containing chemotherapy or within 12 months of neoadjuvant or adjuvant treatment with platinum-containing chemotherapy. In specific embodiments, the patient's tumor expresses PD-L1 [Combined Positive Score (CPS)≥1].

In one embodiment, the invention comprises a method of treating unresectable or metastatic, microsatellite instability-high (MSI-H) or mismatch repair deficient solid tumors in a human patient comprising administering a formulation of the invention to the patient. In specific embodiments, the patient had disease progression following prior anti-cancer treatment.

In one embodiment, the invention comprises a method of treating unresectable or metastatic, microsatellite instability-high (MSI-H) or mismatch repair deficient colorectal cancer in a human patient comprising administering a formulation of the invention. In specific embodiments, the patient had disease progression following prior treatment with a fluoropyrimidine, oxaliplatin, and irinotecan.

In one embodiment, the invention comprises a method of treating recurrent locally advanced or metastatic gastric cancer in a human patient comprising administering a formulation of the invention to the patient.

In one embodiment, the invention comprises a method of treating recurrent locally advanced or metastatic gastroesophageal junction adenocarcinoma in a human patient comprising administering a formulation of the invention to the patient. In specific embodiments, the patient's tumor expresses PD-L1 [Combined Positive Score (CPS) 1]. In specific embodiments, the patient has disease progression on or after two or more prior lines of therapy including fluoropyrimidine- and platinum-containing chemotherapy. In specific embodiments, the patient has disease progression on or after two or more prior lines of therapy including HER2/neu-targeted therapy.

In one embodiment, the invention comprises a method of treating recurrent locally advanced or metastatic cervical cancer in a human patient comprising administering a formulation of the invention to the patient. In specific embodiments, the patient's tumor expresses PD-L1 [Combined Positive Score (CPS)≥1].

In one embodiment, the invention comprises a method of treating hepatocellular carcinoma in a human patient comprising administering a formulation of the invention to the patient. In one embodiment, the invention comprises a method of treating recurrent locally advanced or metastatic merkel cell carcinoma in a human patient comprising administering a formulation of the invention to the patient. In one embodiment, the invention comprises a method of treating recurrent or metastatic cutaneous squamous cell carcinoma in a human patient comprising administering a formulation of the invention to the patient.

In one embodiment, the invention comprises a method of treating advanced renal cell carcinoma in a human patient comprising administering a formulation of the invention to the patient and axitinib. In one embodiment, the invention comprises a method of treating advanced endometrial carcinoma in a human patient comprising administering a formulation of the invention to the patient and lenvatinib. In one embodiment, the endometrial carcinoma is not MSI-H or dMMR.

In one embodiment, the invention comprises a method of treating cancer in a human patient comprising administering a formulation of the invention to the patient, wherein the patient has a cancer selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer.

In one embodiment, the invention comprises a method of treating small cell lung cancer in a human patient comprising administering a formulation of the invention to the patient.

In one embodiment, the invention comprises a method of treating non-Hodgkin lymphoma in a human patient comprising administering a formulation of the invention to the patient. In specific embodiments, the non-Hodgkin lymphoma is mediastinal large B-cell lymphoma. In specific embodiments, the non-Hodgkin lymphoma is diffuse large B-cell lymphoma.

In one embodiment, the invention comprises a method of treating breast cancer in a human patient comprising administering a formulation of the invention to the patient. In certain embodiments, the breast cancer is triple negative breast cancer. In certain embodiments, the breast cancer is ER+/HER2-breast cancer.

In one embodiment, the invention comprises a method of treating nasopharyngeal cancer in a human patient comprising administering a formulation of the invention to the patient.

In one embodiment, the invention comprises a method of treating thyroid cancer in a human patient comprising administering a formulation of the invention to the patient.

In one embodiment, the invention comprises a method of treating salivary cancer in a human patient comprising administering a formulation of the invention to the patient.

Antagonist anti-PD-1 antibodies or antibody fragments can also be used to prevent or treat infections and infectious disease. Thus, the invention provides a method for treating chronic infection in a mammalian subject comprising administering an effective amount of a formulation of the invention to the subject. In some specific embodiments of this method, the formulation is administered to the subject by subcutaneous administration.

These agents can be used alone, or in combination with vaccines, to stimulate the immune response to pathogens, toxins, and self-antigens. The antibodies or antigen-binding fragment thereof can be used to stimulate immune response to viruses infectious to humans, including but not limited to: human immunodeficiency viruses, hepatitis viruses class A, B and C, Epstein Barr virus, human cytomegalovirus, human papilloma viruses, and herpes viruses. Antagonist anti-PD-1 antibodies or antibody fragments can be used to stimulate immune response to infection with bacterial or fungal parasites, and other pathogens. Viral infections with hepatitis B and C and HIV are among those considered to be chronic viral infections.

The formulations of the invention may be administered to a patient in combination with one or more “additional therapeutic agents”. The additional therapeutic agent may be a biotherapeutic agent (including but not limited to antibodies to VEGF, EGFR, Her2/neu, VEGF receptors, other growth factor receptors, CD20, CD40, CD-40L, OX-40, 4-1BB, and ICOS), a growth inhibitory agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib. In still further embodiments, the additional therapeutic agent is a STING agonist. In one embodiment, the additional therapeutic agent is Coxsakievirus CVA21.

Suitable routes of administration for the additional therapeutic agents may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intraarterial or intravenous injection.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g. biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See, e.g., Remington's Pharmaceutical Sciences and U.S. Pharmacopeia: National Formulary, Mack Publishing Company, Easton, PA (1984); Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY.

A pharmaceutical antibody formulation can be administered by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J Med. 349:427-434; Herold et al. (2002) New Engl. J Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

In certain embodiments, dosing will comprise administering to a subject escalating doses of 1.0, 3.0, and 10 mg/kg of the pharmaceutical formulation, i.e, a formulation comprising pembrolizumab, over the course of treatment. The formulation comprising pembrolizumab can be a reconstituted liquid formulation, or it can be a liquid formulation not previously lyophilized. Time courses can vary, and can continue as long as desired effects are obtained. In certain embodiments, dose escalation will continue up to a dose of about 10 mg/kg. In certain embodiments, the subject will have a histological or cytological diagnosis of melanoma, or other form of solid tumor, and in certain instances, a subject may have non-measurable disease. In certain embodiments, the subject will have been treated with other chemotherapeutics, while in other embodiments, the subject will be treatment naïve.

In yet additional embodiments, the dosing regimen will comprise administering a dose of 1, 3, or 10 mg/kg of any of the pharmaceutical formulations described herein (i.e, a formulation comprising pembrolizumab), throughout the course of treatment. For such a dosing regimen, the interval between doses will be about 14 days (±2 days). In certain embodiments, the interval between doses will be about 21 days (±2 days).

In certain embodiments, the dosing regimen will comprise administering a dose of from about 0.005 mg/kg to about 10 mg/kg, with intra-patient dose escalation. In certain embodiments, a dose of 5 mg/kg or 10 mg/kg will be administered at intervals of every 3 weeks, or every 2 weeks. In yet additional embodiments, a dose of 3 mg/kg will be administered at three week intervals for melanoma patients or patients with other solid tumors. In these embodiments, patients should have non-resectable disease; however, patients may have had previous surgery.

In certain embodiments, a subject will be administered a 30 minute IV infusion of any of the pharmaceutical formulations described herein. In certain embodiments for the escalating dose, the dosing interval will be about 28 days ((±1 day) between the first and second dose. In certain embodiments, the interval between the second and third doses will be about 14 days (±2 days). In certain embodiments, the dosing interval will be about 14 days (±2 days), for doses subsequent to the second dose. In certain embodiments, the dosing interval will be about 3 weeks, for doses subsequent to the second dose. In certain embodiments, the dosing interval will be about 6 weeks, for doses subsequent to the second dose.

In certain embodiments, the use of cell surface markers and/or cytokine markers, as described in WO2012/018538 or WO2008/156712 will be used in bioassays for monitoring, diagnostic, patient selection, and/or treatment regimens involving blockade of the PD-1 pathway. Subcutaneous administration may performed by injected using a syringe, or using other injection devices (e.g. the Inject-ease® device); injector pens; or needleless devices (e.g. MediJector and BioJector®).

Embodiments of the invention also include one or more of the biological formulations described herein (i) for use in, (ii) for use as a medicament or composition for, or (iii) for use in the preparation of a medicament for: (a) therapy (e.g., of the human body); (b) medicine; (c) induction of or increasing of an antitumor immune response (d) decreasing the number of one or more tumor markers in a patient; (e) halting or delaying the growth of a tumor or a blood cancer; (f) halting or delaying the progression of PD-1-related disease; (g) halting or delaying the progression of cancer; (h) stabilization of PD-1-related disease; (i) inhibiting the growth or survival of tumor cells; (j) eliminating or reducing the size of one or more cancerous lesions or tumors; (k) reduction of the progression, onset or severity of PD-1-related disease; (1) reducing the severity or duration of the clinical symptoms of PD-1-related disease such as cancer (m) prolonging the survival of a patient relative to the expected survival in a similar untreated patient n) inducing complete or partial remission of a cancerous condition or other PD-1 related disease, or o) treatment of cancer.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J Immunol. 160:1029; Tang et al. (1999) J. Biol. Chem. 274:27371-27378; Baca et al. (1997) J Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J Immunol. 146:169-175; Gibellini et al. (1998) J. Immunol. 160:3891-3898; Hsing and Bishop (1999) J. Immunol. 162:2804-2811; Everts et al. (2002) J Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY). Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1: Evaluation of Interaction Between Pembrolizumab and Recombinant Human Hyaluronidase

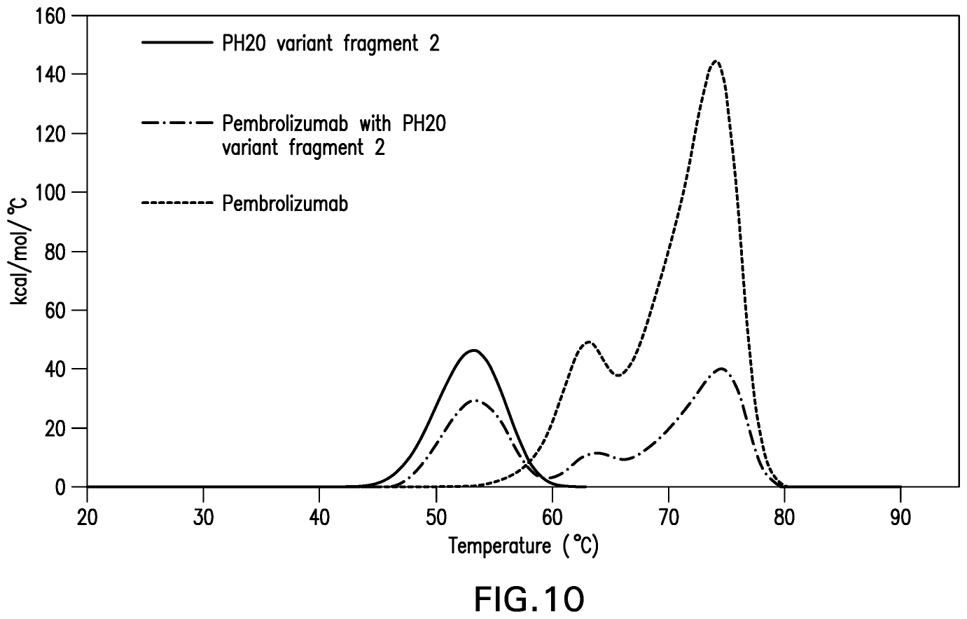

An initial interaction evaluation between pembrolizumab and recombinant human hyaluronidase enzyme, PH20 variant fragment 2 was performed using a Valerian-Plotnikov differential scanning calorimetry. A co-formulation was prepared by diluting pembrolizumab drug substance (165 mg/mL) and enzyme drug substance [solution comprised of PH20 variant fragment 2 (10 mg/mL, ~145 k IU/mg) and sodium chloride (145 mM) in histidine buffer (20 mM)] to 1 mg/mL pembrolizumab and 1 mg/mL of recombinant human hyaluronidase enzyme, PH20 variant fragment 2 as target concentration in the solution with a process diluent solution [7% w/v (70 mg/mL) sucrose, 0.02% w/v (0.2 mg/mL) polysorbate-80 (PS80), 10 mM (1.49 mg/mL) L-methionine in 10 mM histidine buffer (pH 5.5)]. The thermal stability profile of pembrolizumab and enzyme in the same formulation was compared to single entity in the same buffer matrix as shown in FIG. 10. Melting temperature of pembrolizumab and PH20 variant fragment 2 in the co-formulation are identical with single entity in the same buffer matrix, as shown in Table 4.

TABLE 4
Melting and Onset temperature of Pembrolizumab
and PH20 variant fragment 2.

| | | | PH20 variant |
|---|---|---|---|
| | Pembrolizumab | Pembrolizumab | fragment 2 |
| | Melting | Melting | Melting |
| Sample ID | Temperature 1 | Temperature 2 | Temperature |
| PH20 variant fragment 2 | N/A | N/A | 53 |
| Pembrolizumab with | 64 | 75 | 54 |
| PH20 variant fragment 2 | | | |
| Pembrolizumab | 64 | 74 | N/A |

### Example 2: Materials and Analytical Methods

HP-IEX: High performance ion-exchange chromatography (HP-IEX) was used to assess the charge profile. An ion exchange HPLC method was performed using a Dionex ProPac WCX-10 column and a UV detector at 280 nm. Samples were diluted in purified water, and 80 μg were injected for analysis. The mobile phase used for the IEX analysis was a gradient of the following mobile phases (mobile phase A: 24 mM MES, pH 6.1, 4% acetonitrile (v/v); mobile phase B: 20 mM phosphate, 95 mM NaCl, pH 8, 4% acetonitrile (v/v). The main peak is the major component of the chromatogram and it serves as a control for the characterization of acidic and basic variants. Acidic variants elute earlier than main peak and the main cause of the formation of acidic variants is due to the deamidation of the Asn in main peak and the presence of sialic acid compared to main peak. Basic variants elute later than main peak and the main cause of the formation of basic variants is due to the incomplete removal of C-terminal Lys from the main peak. Other causes are incomplete cyclization of the N-terminal glutamine (Gln) to pyroGlu of the light chain or heavy chain or both and also due to the Isomerization of Asp in the main peak to isoAsp.

UP-SEC: Purity of the sample was assessed by size exclusion chromatography (SEC) in which the percentage of monomer was determined, as well as the percentages of high molecular weight species (HMW) and late eluting peaks (LMW species). The presence of HMW species indicates protein aggregates and the presence of LMW species indicate protein fragments. Ultra Performance—Size Exclusion Chromatography (UP-SEC) was performed by diluting the samples to 5.0 mg/mL with sample diluent (mobile phase, 50 mM Phosphate, 450 mM Arginine mono HCl, pH 7±0.2). The diluted samples were injected (6 μL) into a UPLC equipped with a Waters BEH200 SEC column and a UV detector. Proteins in the sample were separated by size and detected by UV absorption at 280 nm.

A350: UV absorption at 350 nm was measured using 96 well plate Spectramax reader as an indication of turbidity. The absorption readings were blanked against empty plate reading and normalized for sample pathlength.

HP-HIC: High performance hydrophobic interaction chromatography (HP-HIC) was used to assess oxidized products from the non-oxidized molecule. The percentage of pre-peaks, determined to be oxidized species comprising heavy chain Met105 oxidation on one heavy chain by previous analytical characterization, as well as the percentage of the main and percentage of the post peaks were determined. A HP-HIC method was performed by diluting the sample to 5.0 mg/mL in purified water. The sample was then injected (10 μL) into an HPLC equipped with a Tosoh Phenyl-5PW column and a UV detector at 280 nm. For the HIC analysis a mobile phase containing a gradient of the following components (mobile phase A: 5 mM sodium phosphate in 2% acetonitrile, pH 7.0; mobile phase B: 400 mM ammonium sulfate, 5 mM sodium phosphate in 2% acetonitrile, pH 6.9;) was used.

Antibody concentration was determined using a gravimetric method where the sample was diluted with water and UV absorption at 280 nm was measured using 96 well plate Spectramax reader. pH was measured using Rapid_pH automated pH meter in a 96 well plate. pH meter was calibrated using pH 4.0, pH 7.0 and pH 10.0 calibration standards.

Density was measured using Anton Paar DMA density meter. Viscosity was measured as per USP <913> technique, using MiniVisII viscometer by Grabner Instruments. The viscosity at 5 °C and 20 °C was measured using respective densities at 5 °C and 20 °C.

### Example 3: Evaluation of the Stability of Pembrolizumab Formulations with Recombinant Human Hyaluronidase PH20 Variant Fragment 2

An initial formulation study was performed to evaluate the stability of formulations comprising of pembrolizumab (25-200 mg/mL) and recombinant human hyaluronidase enzyme, PH20 variant fragment 2 (about 125-5000 U/mL). Formulations were prepared by combining Pembrolizumab drug substance (165 mg/mL) and enzyme drug substance [solution comprised of PH20 variant fragment 2 (10 mg/mL, ~145 k IU/mg) and sodium chloride (145 mM) in histidine buffer (20 mM)] with subsequent dilution where necessary with a process diluent solution [7% w/v (70 mg/mL) sucrose, 0.02% w/v (0.2 mg/mL) polysorbate-80 (PS80), 10 mM (1.49 mg/mL) L-methionine in 10 mM histidine buffer (pH 5.5)].

Test formulations (AK01-AK15) of pembrolizumab and PH20 variant fragment 2 were prepared in PETG bottles (30 or 125 mL) at the volumes outlined in Table 5. The compounding order was PH20 variant fragment 2, pembrolizumab then placebo (where required). All formulations were filtered using 0.22 μm PES filter (Corning REF 431096), and filled into 6R vials with a 5 mL fill volume. Samples were staged on stability (protected from light) at 2-8 °C [as used herein and throughout the Examples, the term “5 °C” is used interchangeably with “2-8 °C”, which indicates 5 °C+3 °C (standard deviation)], 25 and 40 °C for up to 6 months to assess the formulation's thermal stability. Formulations were evaluated for turbidity (A350), pH, protein concentration (A280), soluble aggregates (UP-SEC), charge variants (HP-IEX), Methionine[105]oxidation (HP-HIC) and enzyme activity (turbidimetric assay).

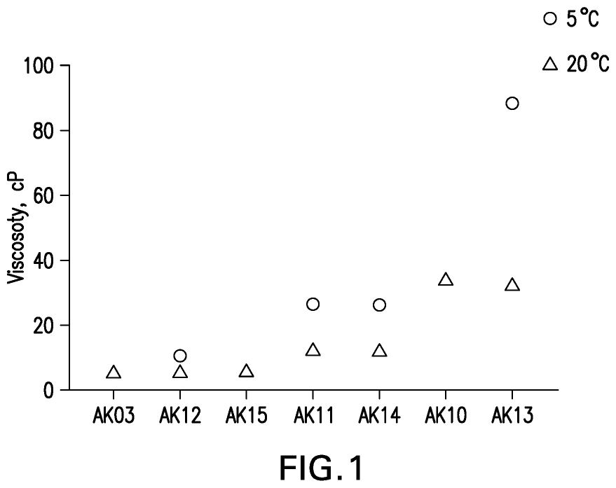

Each of the test formulations in Table 5 were visually inspected for changes in coloration or precipitate formation (data not shown). Formulations AK04-AK06 are enzyme only formulations and were not staged on stability. These formulations were tested for enzyme activity only. The physicochemical properties (density and viscosity) of formulations AK03 and AK10-AK15 were determined and the results are shown in Table 6. The density of each formulation tested was comparable to the analogous pembrolizumab-only formulation (no PH20 variant fragment 2), supporting the compatibility of PH20 variant fragment 2 and pembrolizumab proteins. Furthermore, as shown in Table 6 and FIG. 1, the presence of PH20 variant fragment 2 (at about 2000 or 5000 U/mL) in the pembrolizumab formulations (100, 130 or 165 mg/ml) had no impact on the resulting temperature-dependent viscosities (5 and 20 °C).

TABLE 5
Pembrolizumab + PH20 variant fragment 2 Formulations.

| | | | PH20 variant | | | | |
|---|---|---|---|---|---|---|---|
| | Form- | Pembro | fragment 2 | | | | |
| | ulation | Concentration, | Concentration, | | | | Anti- |
| # | Name | mg/mL | mg/mL | Buffer | Stabilizer | Surfactant | oxidant |
| 1 | AK 01 | 165 | 0 | 10 mM | 7% Sucrose | 0.02% PS-80 | 10 mM |
| | | | | Histidine, pH | | | Methionine |
| | | | | 5.5 | | | |
| 2 | AK 02 | 130 | 0 | 10 mM | 7% Sucrose | 0.02% PS-80 | 10 mM |
| | | | | Histidine, pH | | | Methionine |
| | | | | 5.5 | | | |
| 3 | AK 03 | 100 | 0 | 10 mM | 7% Sucrose | 0.02% PS-80 | 10 mM |
| | | | | Histidine, pH | | | Methionine |
| | | | | 5.5 | | | |
| 4 | AK 04 | 0 | 0.006 | 10 mM | 7% Sucrose | 0.02% PS-80 | 10 mM |
| | | | | Histidine, pH | | | Methionine |
| | | | | 5.5 | | | |
| 5 | AK 05 | 0 | 0.012 | 10 mM | 7% Sucrose | 0.02% PS-80 | 10 mM |
| | | | | Histidine, pH | | | Methionine |
| | | | | 5.5 | | | |
| 6 | AK 06 | 0 | 0.030 | 10 mM | 7% Sucrose | 0.02% PS-80 | 10 mM |
| | | | | Histidine, pH | | | Methionine |
| | | | | 5.5 | | | |
| 7 | AK 07 | 165 | 0.024 | 10 mM | 7% Sucrose | 0.02% PS-80 | 10 mM |
| | | | | Histidine, pH | | | Methionine |
| | | | | 5.5 | | | |
| 8 | AK 08 | 130 | 0.024 | 10 mM | 7% Sucrose | 0.02% PS-80 | 10 mM |
| | | | | Histidine, pH | | | Methionine |
| | | | | 5.5 | | | |
| 9 | AK 09 | 100 | 0.024 | 10 mM | 7% Sucrose | 0.02% PS-80 | 10 mM |
| | | | | Histidine, pH | | | Methionine |
| | | | | 5.5 | | | |
| 10 | AK 10 | 165 | 0.012 | 10 mM | 7% Sucrose | 0.02% PS-80 | 10 mM |
| | | | | Histidine, pH | | | Methionine |
| | | | | 5.5 | | | |
| 11 | AK 11 | 130 | 0.012 | 10 mM | 7% Sucrose | 0.02% PS-80 | 10 mM |
| | | | | Histidine, pH | | | Methionine |
| | | | | 5.5 | | | |
| 12 | AK 12 | 100 | 0.012 | 10 mM | 7% Sucrose | 0.02% PS-80 | 10 mM |
| | | | | Histidine, pH | | | Methionine |
| | | | | 5.5 | | | |
| 13 | AK 13 | 165 | 0.030 | 10 mM | 7% Sucrose | 0.02% PS-80 | 10 mM |
| | | | | Histidine, pH | | | Methionine |
| | | | | 5.5 | | | |
| 14 | AK 14 | 130 | 0.030 | 10 mM | 7% Sucrose | 0.02% PS-80 | 10 mM |
| | | | | Histidine, pH | | | Methionine |
| | | | | 5.5 | | | |
| 15 | AK 15 | 100 | 0.030 | 10 mM | 7% Sucrose | 0.02% PS-80 | 10 mM |
| | | | | Histidine, pH | | | Methionine |
| | | | | 5.5 | | | |

TABLE 6
Density and Viscosity of Formulations at 5 °C and 20 °C

| | Density at 5 | Density at 20 | Viscosity at | Viscosity at |
|---|---|---|---|---|
| Formulation | °C, g/cm³ | °C, g/cm³ | 5 °C, cP | 20 °C, cP |
| AK 03 | NT | 1.05635 | NT | 5.789 |
| AK 10 | NT | 1.07480 | NT | 34.30 |
| AK 11 | 1.06837 | 1.06530 | 26.53 | 12.68 |
| AK 12 | 1.05945 | 1.05656 | 10.66 | 5.832 |
| AK 13 | 1.07710 | 1.07536 | 88.27 | 32.46 |
| AK 14 | 1.06844 | 1.06532 | 26.36 | 12.43 |
| AK 15 | NT | 1.05634 | NT | 6.031 |

NT = Not Tested

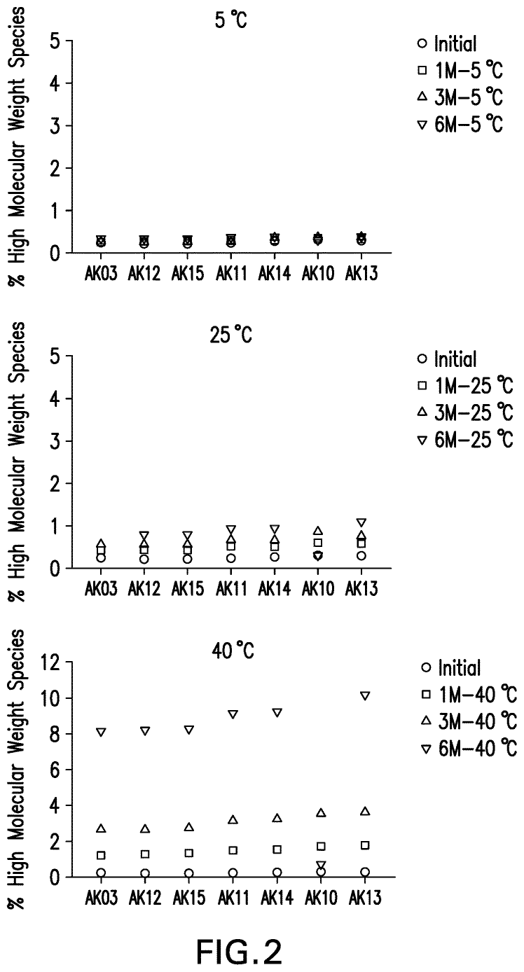

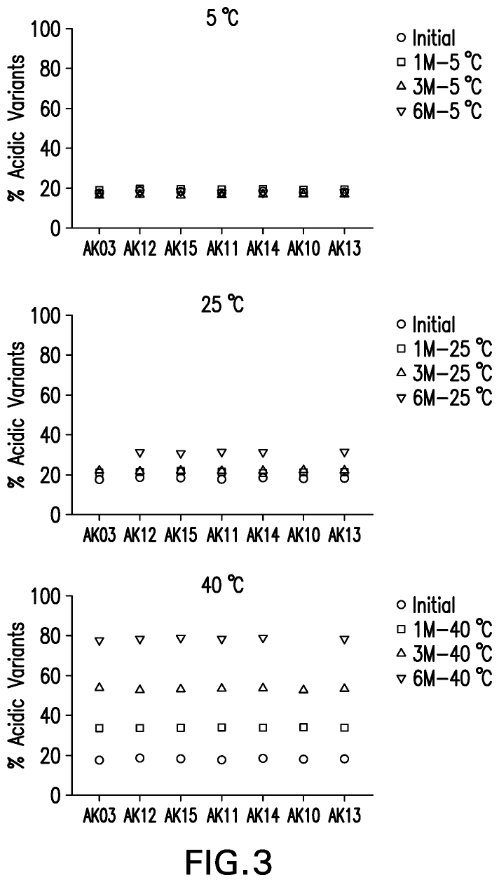

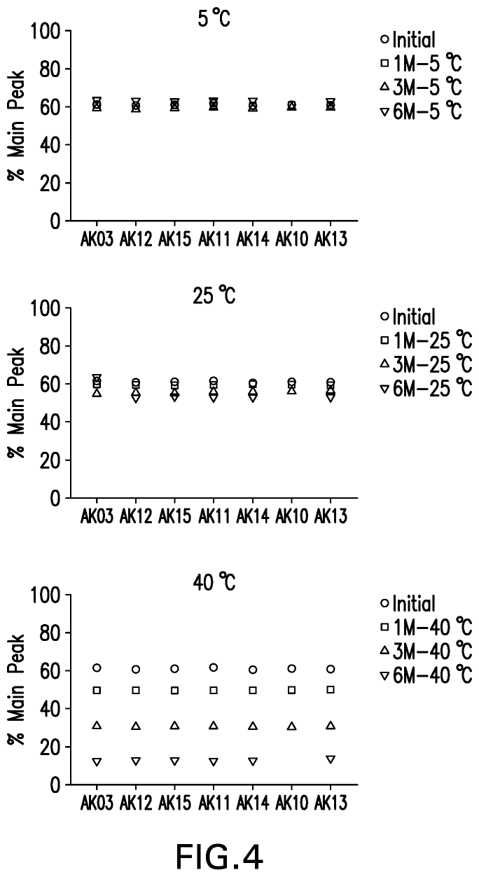

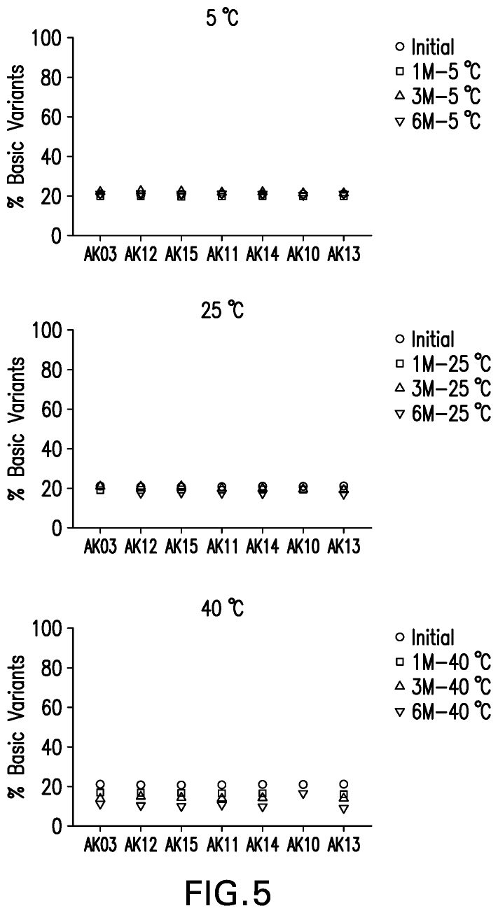

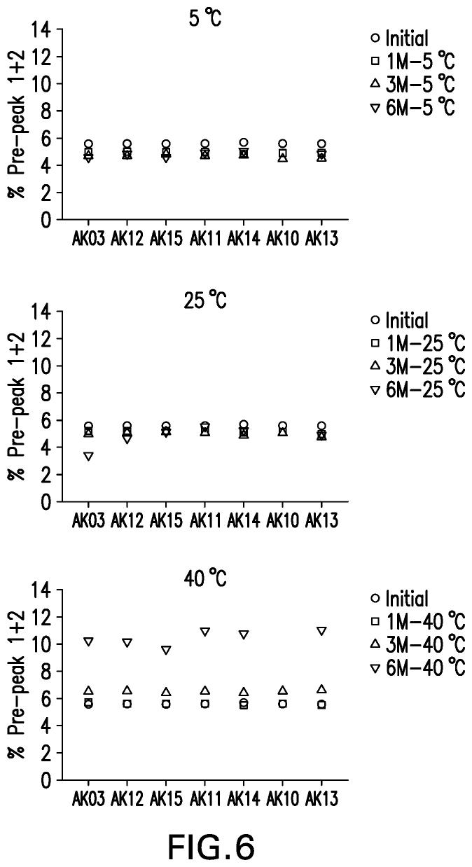

All formulations were considered stable at the 5 °C storage condition through the 6 month stability period as supported by the pH, protein (pembrolizumab) concentration, UP-SEC, HIP-TEX, HIC, turbidity and enzyme activity results (Tables 7-12). At 25 °C, no changes were observed in pH, pembrolizumab concentration, turbidity, charge variants (1EX), Met[105]oxidation, or enzyme activity through the stability period. As shown in FIG. 2, slight increase in soluble aggregates (% High Molecular Weight Species, % HMWS), and corresponding decrease in % monomer, were observed via UP-SEC over the time period tested when compared to the control (AK03, PH20 variant fragment 2-free formulation). More marked changes in soluble aggregates (% HMWS) were observed at 40 °C for all formulations tested (FIG. 2), with the largest changes observed as the concentration of pembrolizumab increased (100 mg/mL→165 mg/mL) Turbidity (A350) results (Table 7) for all formulations also corroborated the UP-SEC results indicating a decrease in physical stability of all formulations stored at 40 °C for >1 month. Additionally, at 40 °C, more pronounced changes were observed in charge variants (FIGS. 3-5) and methionine[105]oxidation (FIG. 6) for all formulations tested (Tables 10 and 12), however the presence of PH20 variant fragment 2 up to about 5000 U/mL in the formulation does not impact any of the formulation attributes tested when compared to the control (AK03, PH20 variant fragment 2-free formulation). No changes in pembrolizumab concentration or formulation pH were observed through the 6-month stability period at 5, 25 or 40 °C, demonstrating the chemical stability of pembrolizumab in all formulations including the formulations with PH20 variant fragment 2 (about 2000-5000 U/mL).

TABLE 7
Turbidity Results for Formulations AK01-AK15.

| | | Stability Time-Temperature | | | | | | | | | |
|---|---|---|---|---|---|---|---|---|---|---|---|
| Test | Form. | Initial | 1M/5 C | 3M/5 C | 6M/5 C | 1M/25 C | 3M/25 C | 6M/25 C | 1M/40 C | 3M/40 C | 6M/40 C. |
| Turbidity | AK 01 | 0.236 | NS | | | | | | | | |
| (A350) | AK 02 | 0.141 | | | | | | | | | |
| | AK 03 | 0.097 | 0.122 | 0.121 | 0.115 | 0.130 | 0.133 | NS | 0.150 | 0.202 | 0.440 |
| | AK 07 | 0.102 | NS | | | | | | | | |
| | AK 08 | 0.140 | | | | | | | | | |
| | AK 09 | 0.116 | | | | | | | | | |
| | AK 10 | 0.168 | 0.176 | 0.177 | NS | 0.183 | 0.199 | NS | 0.219 | 0.298 | NS |
| | AK 11 | 0.142 | 0.148 | 0.144 | 0.142 | 0.154 | 0.164 | 0.170 | 0.219 | 0.246 | 0.526 |
| | AK 12 | 0.115 | 0.123 | 0.122 | 0.117 | 0.129 | 0.134 | 0.142 | 0.154 | 0.201 | 0.447 |
| | AK 13 | 0.174 | 0.179 | 0.178 | 0.171 | 0.186 | 0.199 | 0.205 | 0.184 | 0.302 | 0.635 |
| | AK 14 | 0.143 | 0.149 | 0.148 | 0.143 | 0.159 | 0.162 | 0.172 | 0.182 | 0.248 | 0.536 |
| | AK 15 | 0.115 | 0.126 | 0.123 | 0.119 | 0.133 | 0.137 | 0.143 | 0,155 | 0.205 | 0.449 |

NT = Not Tested;
NS = Not staged on Stability

TABLE 8
pH Data for Formulations AK-01-AK15.

| | | Stability Time-Temperature | | | | | | | | | |
|---|---|---|---|---|---|---|---|---|---|---|---|
| Test | Form. | Initial | 1M/5 C | 3M/5 C | 6M/5 C | 1M/25 C | 3M/25 C | 6M/25 C | 1M/40 C | 3M/40 C | 6M/40 C |
| pH | AK 01 | 5.6 | NS | | | | | | | | |
| | AK 02 | 5.6 | | | | | | | | | |
| | AK 03 | 5.5 | 5.6 | 5.7 | NS | 5.6 | 5.6 | NS | 5.6 | 5.6 | NT |
| | AK 07 | 5.6 | NS | | | | | | | | |
| | AK 08 | 5.5 | | | | | | | | | |
| | AK 09 | 5.6 | | | | | | | | | |
| | AK 10 | 5.6 | 5.7 | 5.7 | NS | 5.6 | 5.6 | NS | 5.7 | 5.6 | NS |
| | AK 11 | 5.6 | 5.6 | 5.6 | NT | 5.7 | 5.6 | NT | 5.6 | 5.6 | NT |
| | AK 12 | 5.5 | 5.6 | 5.6 | NT | 5.6 | 5.6 | NT | 5.6 | 5.6 | NT |
| | AK 13 | 5.6 | 5.7 | 5.7 | NT | 5.6 | 5.6 | NT | 5.7 | 5.6 | NT |
| | AK 14 | 5.5 | 5.6 | 5.6 | NT | 5.6 | 5.6 | NT | 5.6 | 5.6 | NT |
| | AK 15 | 5.5 | 5.6 | 5.6 | NI | 5.6 | 5.6 | NT | 5.6 | 5.6 | NT |

NT = Not Tested;
NS = Not staged on Stability

TABLE 9
Pembrolizumab Concentration for Formulations AK01-AK15.

| | | Stability Time-Temperature | | | | | | | | | |
|---|---|---|---|---|---|---|---|---|---|---|---|
| Test | Form. | Initial | 1M/5 C | 3M/5 C | 6M/5 C | 1M/25 C | 3M/25 C | 6M/25 C | 1M/40 C | 3M/40 C | 6M/40 C |
| Ab | AK 01 | 163.9 | NS | | | | | | | | |
| Concen- | AK 02 | 132.5 | | | | | | | | | |
| tration | AK 03 | 99.7 | 92.9 | 97.8 | NS | 97.3 | 94.2 | NS | 87.3 | 104.9 | NT |
| (mg/mL) | AK 07 | 158.7 | NS | | | | | | | | |
| | AK 08 | 124.5 | | | | | | | | | |
| | AK 09 | 97.9 | | | | | | | | | |
| | AK 10 | 167.2 | 161.9 | 162.4 | NS | 148.3 | 165.1 | NS | 156.8 | 162.8 | NS |
| | AK 11 | 126.3 | 127.4 | 130.3 | NT | 114.7 | 128.5 | NT | 125.9 | 130.5 | NT |
| | AK 12 | 100.1 | 91.3 | 98.3 | NT | 88.7 | 101.0 | NT | 89.6 | 102.3 | NT |
| | AK 13 | 158.5 | 162.3 | 161.9 | NT | 152.9 | 158.9 | NT | 148.4 | 159.3 | NT |
| | AK 14 | 127.4 | 110.6 | 128.5 | NT | 134.4 | 129.3 | NT | 122.3 | 126.6 | NT |
| | AK 15 | 94.7 | 92 | 92.0 | NT | 87.7 | 107.4 | NT | 99.6 | 99.6 | NT |

NT = Not Tested,
NS = Not staged on Stability

TABLE 10
Methionine[105] Oxidation Results for Formulations AK01-AK15.

| | | Stability Time-Temperature | | | | | | | | | |
|---|---|---|---|---|---|---|---|---|---|---|---|
| Test | Form. | Initial | 1M/5 C | 3M/5 C | 6M/5 C | 1M/25 C | 3M/25 C | 6M/25 C | 1M/40 C | 3M/40 C | 6M/40 C |
| Pre-peak | AK 01 | 5.7 | NS | | | | | | | | |
| 1 & 2 | AK 02 | 5.7 | | | | | | | | | |
| (%) | AK 03 | 5.6 | 5.0 | 4.8 | 4.5 | 5.2 | 5.1 | NS | 5.7 | 6.6 | 10.2 |
| | AK 07 | 5.6 | NS | | | | | | | | |
| | AK 08 | 5.6 | | | | | | | | | |
| | AK 09 | 5.7 | | | | | | | | | |
| | AK 10 | 5.6 | 4.9 | 4.6 | NS | 5.1 | 5.2 | NS | 5.6 | 6.6 | NS |
| | AK 11 | 5.6 | 5.0 | 4.8 | 4.9 | 5.1 | 5.2 | 5.5 | 5.6 | 6.6 | 10.9 |
| | AK 12 | 5.6 | 5.0 | 4.8 | 4.8 | 5.2 | 5.2 | 4.6 | 5.6 | 6.6 | 10.1 |
| | AK 13 | 5.6 | 4.9 | 4.6 | 4.8 | 5.0 | 4.9 | 5.1 | 5.5 | 6.7 | 10.9 |
| | AK 14 | 5.7 | 4.9 | 4.8 | 5.0 | 5.1 | 5.0 | 5.1 | 5.5 | 6.5 | 10.7 |
| | AK 15 | 5.6 | 5.0 | 4.9 | 4.5 | 5.2 | 5.2 | 5.1 | 5.6 | 6.5 | 9.5 |

NS = Not staged on Stability

TABLE 11
UP-SEC Results for Formulations AK01-AK15.

| | | Stability Time-Temperature | | | | | | | | | |
|---|---|---|---|---|---|---|---|---|---|---|---|
| Test | Form. | Initial | 1M/5 C | 3M/5 C | 6M/5 C | 1M/25 C | 3M/25 C | 6M/25 C | 1M/40 C | 3M/40 C | 6M/40 C |
| % HMWS | AK 01 | 0.32 | NS | | | | | | | | |
| | AK 02 | 0.28 | | | | | | | | | |
| | AK 03 | 0.25 | 0.28 | 0.29 | 0.30 | 0.43 | 0.58 | NS | 1.22 | 2.70 | 8.14 |
| | AK 07 | 0.31 | NS | | | | | | | | |
| | AK 08 | 0.28 | | | | | | | | | |
| | AK 09 | 0.22 | | | | | | | | | |
| | AK 10 | 0.31 | 0.34 | 0.38 | NS | 0.60 | 0.93 | NS | 1.73 | 2.70 | NS |
| | AK 11 | 0.24 | 0.30 | 0.33 | 0.33 | 0.52 | 0.69 | 0.90 | 1.49 | 3.15 | 9.13 |
| | AK 12 | 0.22 | 0.27 | 0.31 | 0.30 | 0.44 | 0.58 | 0.76 | 1.29 | 2.73 | 8.16 |
| | AK 13 | 0.30 | 0.33 | 0.38 | 0.34 | 0.58 | 0.82 | 1.06 | 1.78 | 3.72 | 10.17 |
| | AK 14 | 0.27 | 0.30 | 0.40 | 0.34 | 0.51 | 0.69 | 0.91 | 1.54 | 3.29 | 9.20 |
| | AK 15 | 0.22 | 0.27 | 0.29 | 0.30 | 0.43 | 0.59 | 0.76 | 1.33 | 2.80 | 8.20 |
| % Monomer | AK 01 | 99.7 | NS | | | | | | | | |
| | AK 02 | 99.7 | | | | | | | | | |
| | AK 03 | 99.8 | 99.7 | 99.7 | 99.7 | 99.6 | 99.4 | NS | 98.8 | 97.2 | 91.6 |
| | AK 07 | 99.7 | NS | | | | | | | | |
| | AK 08 | 99.7 | | | | | | | | | |
| | AK 09 | 99.8 | | | | | | | | | |
| | AK 10 | 99.7 | 99.7 | 99.6 | NS | 99.4 | 99.1 | NS | 98.3 | 96.2 | NS |
| | AK 11 | 99.8 | 99.7 | 99.7 | 99.7 | 99.5 | 99.3 | 99.1 | 98.5 | 96.8 | 90.7 |
| | AK 12 | 99.8 | 99.7 | 99.7 | 99.7 | 99.6 | 99.4 | 99.2 | 98.7 | 97.2 | 91.6 |
| | AK 13 | 99.7 | 99.7 | 99.6 | 99.7 | 99.3 | 99.2 | 98.9 | 98.2 | 96.2 | 89.6 |
| | AK 14 | 99.7 | 99.7 | 99.6 | 99.7 | 99.5 | 99.3 | 99.1 | 98.4 | 96.6 | 90.6 |
| | AK 15 | 99.8 | 99.7 | 99.7 | 99.7 | 99.5 | 99.4 | 99.2 | 98.7 | 97.1 | 91.6 |
| % LMWS | AK 01 | 0.00 | NS | | | | | | | | |
| | AK 02 | 0.00 | | | | | | | | | |
| | AK 03 | 0.00 | 0.00 | 0.00 | 0.00 | 0.00 | 0.00 | NS | 0.02 | 0.09 | 0.23 |
| | AK 07 | 0.00 | NS | | | | | | | | |
| | AK 08 | 0.00 | | | | | | | | | |
| | AK 09 | 0.00 | | | | | | | | | |
| | AK 10 | 0.00 | 0.00 | 0.00 | NS | 0.00 | 0.00 | NS | 0.02 | 0.09 | NS |
| | AK 11 | 0.00 | 0.03 | 0.00 | 0.00 | 0.00 | 0.00 | 0.02 | 0.02 | 0.08 | 0.21 |
| | AK 12 | 0.00 | 0.03 | 0.00 | 0.00 | 0.00 | 0.00 | 0.02 | 0.02 | 0.08 | 0.23 |
| | AK 13 | 0.00 | 0.00 | 0.00 | 0.00 | 0.09 | 0.00 | 0.02 | 0.02 | 0.08 | 0.20 |
| | AK 14 | 0.00 | 0.00 | 0.00 | 0.00 | 0.00 | 0.00 | 0.02 | 0.02 | 0.09 | 0.23 |
| | AK 15 | 0.00 | 0.01 | 0.00 | 0.00 | 0.09 | 0.00 | 0.01 | 0.02 | 0.09 | 0.23 |
| . | | | | | | | | | | | |

NS = Not staged on Stability

TABLE 12
Charge Variants

| | | Stability Time-Temperature | | | | | | | | | |
|---|---|---|---|---|---|---|---|---|---|---|---|
| Test | Form. | Initial | 1M/5 C | 3M/5 C | 6M/5 C | 1M/25 C | 3M/25 C | 6M/25 C | 1M/40 C | 3M/40 C | 6M/40 C |
| Acidic | AK 01 | 18.2 | NS | | | | | | | | |
| Variants | AK 02 | 18.2 | | | | | | | | | |
| (%) | AK 03 | 17.6 | 19.1 | 17.0 | 16.8 | 21.1 | 22.9 | NS | 33.8 | 54.4 | 77.1 |
| | AK 07 | 17.0 | NS | | | | | | | | |
| | AK 08 | 17.7 | | | | | | | | | |
| | AK 09 | 17.8 | | | | | | | | | |
| | AK 10 | 18.0 | 19.3 | 17.6 | NS | 21.2 | 23.1 | NS | 34.0 | 53.2 | NS |
| | AK 11 | 17.7 | 19.5 | 17.3 | 17.2 | 21.5 | 22.8 | 30.9 | 34.0 | 54.0 | 78.0 |
| | AK 12 | 18.6 | 19.6 | 17.3 | 16.9 | 21.3 | 22.4 | 30.8 | 33.8 | 53.4 | 77.7 |
| | AK 13 | 18.2 | 19.4 | 17.6 | 17.4 | 21.3 | 22.9 | 31.0 | 33.9 | 54.0 | 78.0 |
| | AK 14 | 18.5 | 19.6 | 17.6 | 17.1 | 20.9 | 22.8 | 30.8 | 33.9 | 54.1 | 78.6 |
| | AK 15 | 18.3 | 19.6 | 17.1 | 17.5 | 21.8 | 22.8 | 30.2 | 33.8 | 53.8 | 78.3 |
| Main | AK 01 | 60.6 | NS | | | | | | | | |
| (%) | AK 02 | 60.9 | | | | | | | | | |
| | AK 03 | 61.4 | 60.9 | 60.0 | 62.9 | 59.6 | 55.5 | NS | 49.4 | 31.3 | 12.3 |
| | AK 07 | 62.2 | NS | | | | | | | | |
| | AK 08 | 61.2 | | | | | | | | | |
| | AK 09 | 61.2 | | | | | | | | | |
| | AK 10 | 61.0 | 60.8 | 60.4 | NS | 59.5 | 56.8 | NS | 49.7 | 30.9 | NS |
| | AK 11 | 61.6 | 60.6 | 60.3 | 62.4 | 59.3 | 56.7 | 52.2 | 49.5 | 31.4 | 11.9 |
| | AK 12 | 60.7 | 60.5 | 59.4 | 62.5 | 59.3 | 56.1 | 52.3 | 49.4 | 31.1 | 12.2 |
| | AK 13 | 60.8 | 60.7 | 60.4 | 62.1 | 59.3 | 57.2 | 52.3 | 49.9 | 31.3 | 13.4 |
| | AK 14 | 60.5 | 60.5 | 59.8 | 62.5 | 59.8 | 56.5 | 52.4 | 49.6 | 31.0 | 12.1 |
| | AK 15 | 61.0 | 60.6 | 59.9 | 62.2 | 59.0 | 55.7 | 52.6 | 49.5 | 31.3 | 12.2 |
| Basic | AK 01 | 21.2 | NS | | | | | | | | |
| Variants | AK 02 | 20.8 | | | | | | | | | |
| (%) | AK 03 | 21.1 | 20.0 | 23.0 | 20.3 | 19.2 | 21.6 | NS | 17.0 | 14.3 | 10.6 |
| | AK 07 | 20.8 | NS | | | | | | | | |
| | AK 08 | 21.0 | | | | | | | | | |
| | AK 09 | 21.1 | | | | | | | | | |
| | AK 10 | 20.9 | 19.9 | 22.0 | NS | 19.3 | 20.1 | NS | 16.2 | 15.9 | NS |
| | AK 11 | 20.8 | 19.9 | 22.4 | 20.5 | 19.2 | 20.6 | 16.9 | 16.5 | 14.6 | 10.2 |
| | AK 12 | 20.7 | 19.9 | 23.3 | 20.6 | 19.3 | 21.5 | 17.0 | 16.8 | 15.5 | 10.1 |
| | AK 13 | 21.1 | 19.9 | 22.0 | 20.5 | 19.4 | 19.9 | 16.7 | 16.2 | 14.7 | 8.6 |
| | AK 14 | 20.9 | 20.0 | 22.6 | 20.4 | 19.3 | 20.6 | 16.8 | 16.5 | 14.9 | 9.3 |
| | AK 15 | 20.7 | 19.8 | 23.0 | 20.3 | 19.3 | 21.5 | 17.1 | 16.8 | 14.9 | 9.4 |

NS = Not staged on Stability

1M, 3M and 6M refers to 1 month, 3 months and 6 months, respectively. 5 C, 25 C and 40 C refers to 5 °C, 25 °C and 40 °C, respectively.

### Example 4: Determination of Hyaluronidase Activity in Formulations with Pembrolizumab and Hyaluronidase Variant Fragment

### Hyaluronidase Activity Assay

Enzyme activity within a series of formulations (Table 13) was determined by a turbidimetric assay on a Molecular Devices SpectraMax M5e microplate reader.

TABLE 13
Formulations and Enzyme-only Controls Tested

| | Pembro | PH20 variant fragment 2 |
|---|---|---|
| | Concentration, | Concentration, |
| Formulation* | mg/mL | Units/mL |
| AK 05 | — | 2000 |
| AK 06 | — | 5000 |
| AK 10 | 165 | 2000 |
| AK 11 | 130 | 2000 |
| AK 12 | 100 | 2000 |
| AK 13 | 165 | 5000 |
| AK 14 | 130 | 5000 |
| AK 15 | 100 | 5000 |

*All formulations contain 7% w/v Sucrose, 0.02% w/v PS-80, 10 mM Histidine, 10 mM Methionine at pH 5.5

Calibration curve standards, activity standards and test samples were prepared via dilution with chilled Enzyme Diluent (20 mM sodium phosphate, 77 mM sodium chloride, 0.01% bovine serum albumin (BSA) at pH 7.0 at 25 °C) to the working concentrations outlined in the Table 14 below.

TABLE 14
Example Working Concentrations Used in Activity Assay.

| Sample Type | Working Concentration (Units/mL) |
|---|---|
| Calibration Curve | 20 |
| | 15 |
| | 12 |
| | 10 |
| | 8 |
| | 6 |
| Activity Standard | 15 |
| | 12 |
| | 10 |
| Test Samples | 15 |
| | 12 |
| | 10 |

Samples were kept on ice during preparation. 50 μL of each sample was transferred to a clear-bottom 96-well plate (Plate 1) (Corning 3835) in triplicate. 50 μL of Enzyme Diluent solution was added in dedicated wells on the plate as a blank control. The plate was sealed and incubated at 37 °C for 10 minutes. After incubation, 50 μL of 37 °C Hyaluronic Acid Solution (0.06% hyaluronic acid 300 mM phosphate at pH 5.35 at 37 °C) was added to each well containing solution with a multichannel pipette. The plate was sealed then incubated at 37 °C for exactly 45 minutes with shaking at 600 rpm. Prior to removing Plate 1 from incubation, a second plate (Plate 2) was prepared with 200 μL of Acidic Albumin Solution (24 mM sodium acetate, 79 mM acetic acid, 0.1% BSA at pH 3.75 at 25 °C) in each well creating the same well layout as Plate 1. After the exact 45 minute incubation of Plate 1, 40 μL of solution from each well was removed with a multi-channel pipette and added to the corresponding wells in Plate 2 (containing Acidic Albumin Solution). Plate 2 was incubated at 25 °C for 20 minutes within the plate chamber of the microplate reader. The microplate reader was set to read absorbance at 600 nm after 5 seconds of shaking. After the 20 minute incubation, absorbance at 600 nm was read for each well.

### Generating Calibration Curve

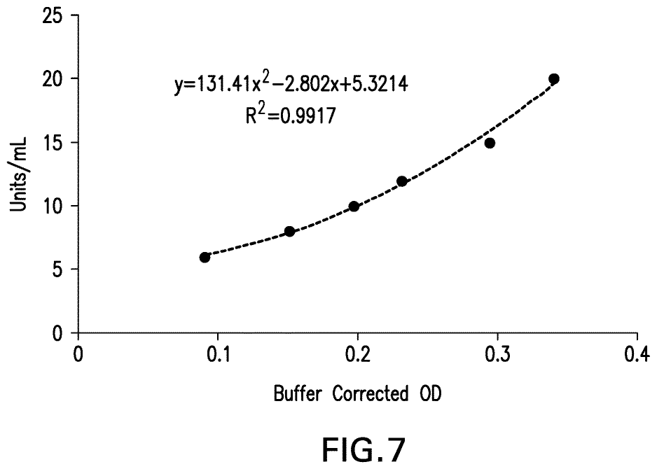

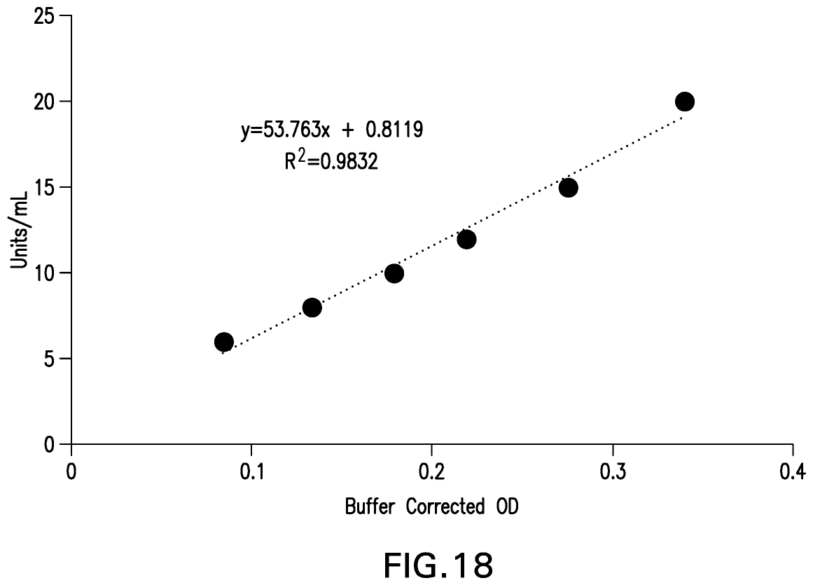

Triplicate absorbance values from calibration curve standards were averaged and subtracted from the average absorbance of the blank. The absolute value of the resulting corrected absorbance was plotted vs the calibration curve standard volumetric activity values (20, 15, 12, 10, 8, and 6 Units/mL, for example). The plot was fit as a second-order polynomial (FIG. 7) and the resulting equation of the fit was used to determine enzymatic activity of the activity standards and test samples. The plot can also be performed with a linear fit, which was applied to the enzyme activity calculations in Examples 5-10 (FIG. 18).

### Calculation of Enzymatic Activity of Standards and Test Samples

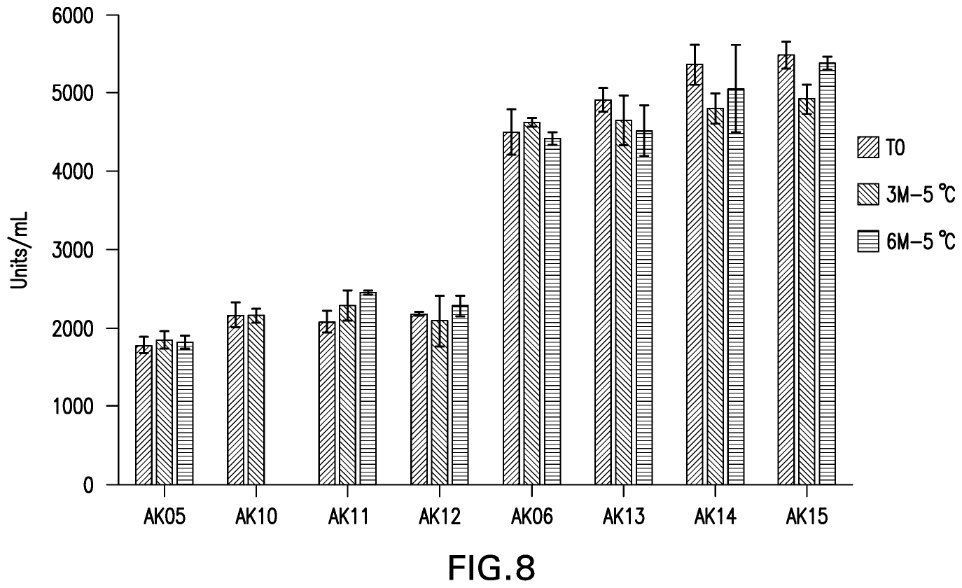

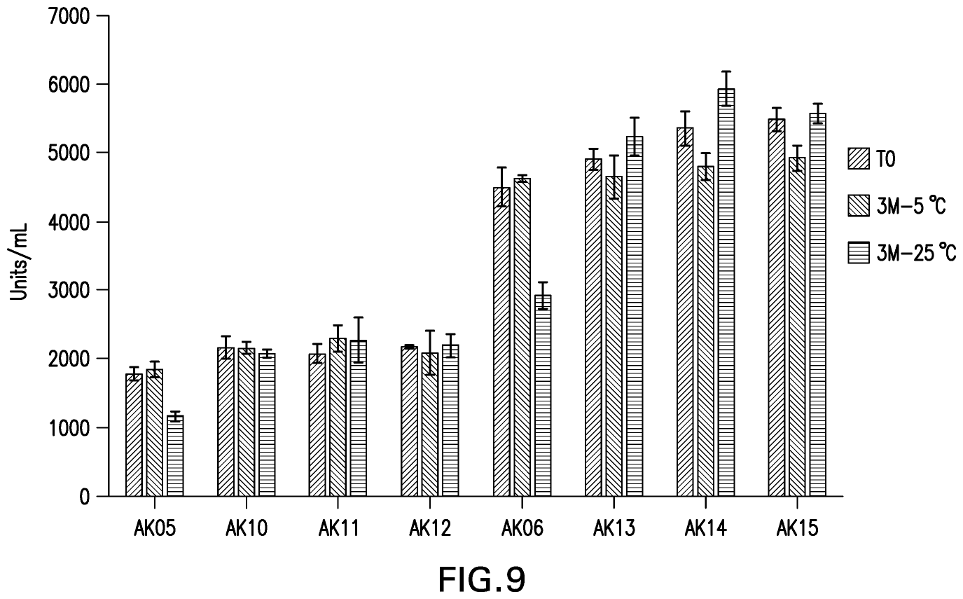

Triplicate absorbance values were averaged and subtracted from the average absorbance of the blank. The absolute value of the resulting corrected absorbance was entered into the fit equation from the calibration curve to determine the assay volumetric activity for the standards and test samples. Any corrected absorbance value that fell outside the calibration curve was discarded. Values were corrected for dilution by multiplying the assay volumetric activity by the dilution factor used to prepare the solutions. (For example, if the estimated enzymatic activity of the formulation or standard stock solution was 1500 Units/mL and was diluted to 15, 12, and 10 Units/mL for analysis, the dilution factors would have been 100, 125, and 150 respectively.) Final volumetric enzyme activity values were averaged and reported in Units/mL as shown in FIG. 8 and FIG. 9.

Enzyme activity has been assessed in the co-formulated (pembrolizumab and PH20 variant fragment 2) and PH20 variant fragment 2 control samples listed in Table 13 following storage at 5 and 25 °C. The enzyme activity in all formulations was retained through 6 month/5 °C storage and comparable to that of the initial samples (FIG. 8). After storage at 3 months/25 °C, enzyme-only controls showed decreased activity relative to samples stored at 5 °C (FIG. 9). Surprisingly, theenzyme activity in co-formulated samples was not impacted through 3 month/25 °C, indicating enhanced enzyme stability and activity in the presence of pembrolizumab.

### Example 5: Evaluation of the Stability of Recombinant Human Hyaluronidase PH20 Variant Fragment 2 with Pembrolizumab in the Formulations Under Stainless Steel (SS) Stress

Test formulations (SS01-SS06) of pembrolizumab and PH20 variant fragment 2 were prepared in PETG bottles (125 mL) with the compositions outlined in Table 15. Formulation SS02, SS04 and SS06 were exposed to SS solid cylinders at room temperature for 24 hours. Formulation SS01, SS03 and SS05 were placed at room temperature for 24 hours as control. All formulations were filtered using 0.22 μm PES filter. Samples were staged on stability (protected from light) at 5 °C up to 6 months and 25 °C for up to 3 months to assess the PH20 variant fragment 2 activity.

TABLE 15
Pembrolizumab + PH20 variant fragment
2 Formulations with and without SS exposure.

| Sample | | |
|---|---|---|
| Name | Composition | Condition |
| SS 01 | 165 mg/mL pembrolizumab, 2000 units/mL PH20 variant | Control |
| | fragment 2, 10 mM L-Methionine, 7% (w/v) Sucrose, | (No SS exposure, 25 °C |
| | 0.02% (w/v) PS80 in 10 mM L-Histidine Buffer, pH 5.5 | for 24 hr) |
| SS 02 | | 1 Day SS exposure |
| | | (SS exposure, 25 °C |
| | | for 24 hr) |
| SS 03 | 130 mg/mL pembrolizumab, 2000 units/mL PH20 variant | Control |
| | fragment 2, 10 mM L-Methionine, 7% (w/v) Sucrose, | (No SS exposure, 25 °C |
| | 0.02% (w/v) PS80 in 10 mM L-Histidine Buffer, pH 5.5 | for 24 hr) |
| SS 04 | | 1 Day SS exposure |
| | | (SS exposure, 25 °C |
| | | for 24 hr) |
| SS 05 | 2000 units/mL PH20 variant fragment 2, 10 mM L- | Control |
| | Methionine, 7% (w/v) Sucrose, 0.02% (w/v) PS80 in 10 | (No SS exposure, 25 °C |
| | mM L-Histidine Buffer, pH 5.5 | for 24 hr) |
| SS 06 | | 1 Day SS exposure |
| | | (SS exposure, 25 °C |
| | | for 24 hr) |

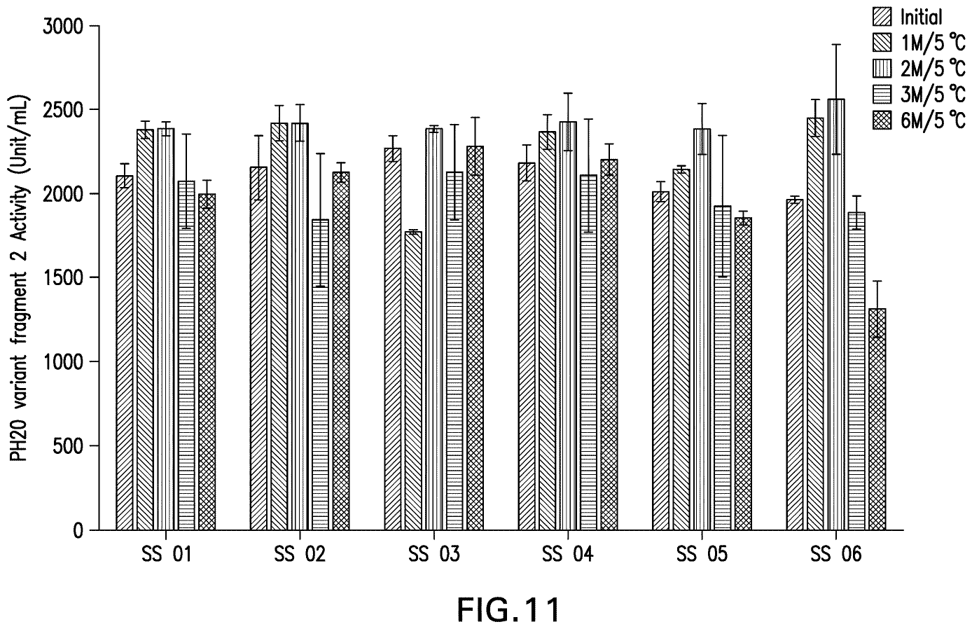

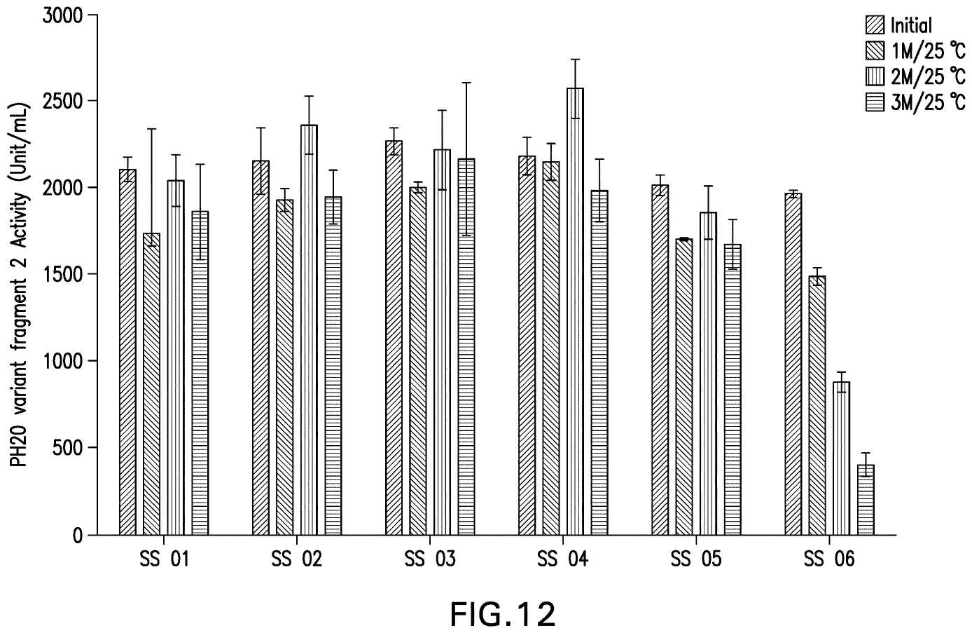

Enzyme activity in co-formulated samples (SS01-SS04) was retained through 6 month/5 °C and 3 month/25 °C storage and comparable to that of the initial samples (FIG. 11 and FIG. 12). After storage at 6 months/S °C and 3 months/25 °C, enzyme-on/y sample with SS stress (SS06) showed decreased activity relative to samples without SS stress (SSOS) (FIG. 11 and FIG. 12). The enzyme activity in co-formulated samples was not impacted through all the temperatures and timepoints, indicating enhanced enzyme stability and activity in the presence of pembrolizumab.

### Example 6: Evaluation of the Stability of Recombinant Human Hyaluronidase PH20 Variant Fragment 2 with Pembrolizumab and Viscosity Surrogate of Pembrolizumab Under Light Stress

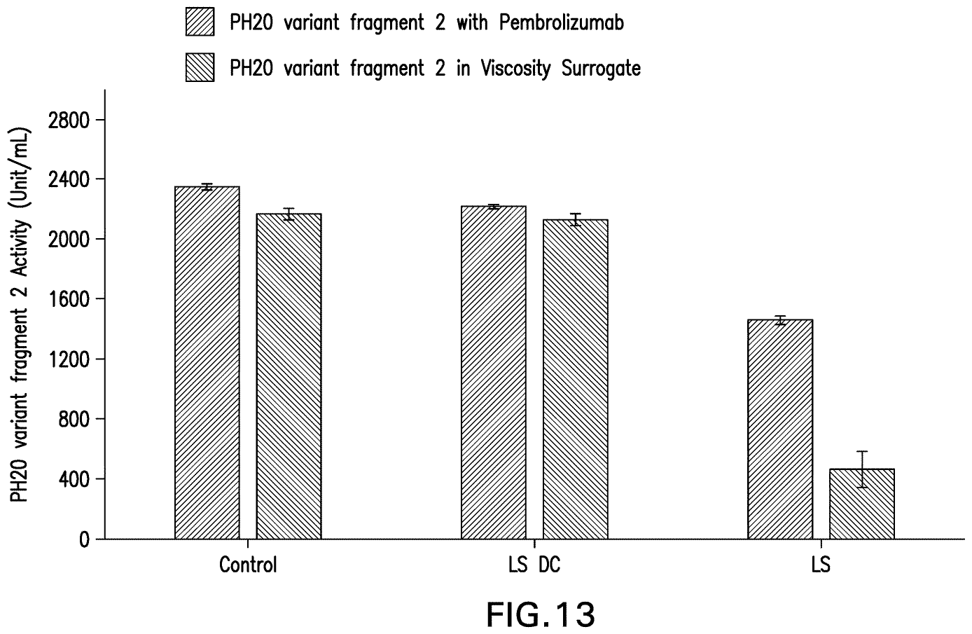

Co-formulated samples were prepared as 165 mg/mL Pembrolizumab, 2000 Units/mL Recombinant Human Hyaluronidase PH20 variant fragment 2 in 700 sucrose, 0.2 mg/mL PS-80, 10 mM methionine in 10 mM Histidine buffer at pH 5.5. To mimic the high viscosity in the solution caused by Pembrolizumab, 2000 Units/mL Recombinant Human Hyaluronidase PH20 variant fragment 2 was formulated in 52% (w/w) Sucrose, 0.02% (w/w) PS-80. Samples were filled into 6R vials with a 5 mL fill volume. 1 vial of each sample was subjected to cumulative light exposure of 300 Klux-hr CWL at equivalent to 0.25×ICH CWL (1 ICH=1200 klux*hr). Additionally, second set of vials for each sample was covered with aluminum foil during the exposure in the light chamber as a dark control. Enzyme activity test were performed to evaluate enzyme stability in the viscosity surrogate under light stress. Both samples show enzyme activity decrease under light stress compared to control samples. Enzyme activity in viscosity surrogate solution decreased more compared to enzyme activity in the presence of Pembrolizumab under the same light stress conditions (FIG. 13).

### Example 7: Evaluation of the Impact of Excipient Concentration on the Stability of Recombinant Human Hyaluronidase PH20 Variant Fragment 2 with Pembrolizumab

Test formulations (ER 01-ER 13) of pembrolizumab (165 mg/mL) and PH20 variant fragment 2 (2000 Units/mL), 10 mM Histidine, pH 5.5 were prepared with the compositions outlined in Table 16 that are designed to differ in their concentrations of excipients polysorbate 80, L-methionine, and sucrose. Samples were incubated at 25 °C for three months to monitor the impact of excipient concentration on enzyme activity.

TABLE 16
Excipient Ranging Study in Pembrolizumab +
PH20 variant fragment 2 Formulations.

| Sample Name | L-Methionine (mM) | Sucrose (% w/v) | PS80 (% w/v) |
|---|---|---|---|
| ER 1 | 30 | 10 | 0.1 |
| ER 2 | 1 | 3 | 0.005 |
| ER 3 | 1 | 10 | 0.1 |
| ER 4 | 30 | 3 | 0.005 |
| ER 5 | 30 | 3 | 0.1 |
| ER 6 | 1 | 3 | 0.005 |
| ER 7 | 1 | 10 | 0.005 |
| ER 8 | 1 | 10 | 0.1 |
| ER 9 | 30 | 10 | 0.005 |
| ER 10 | 1 | 3 | 0.1 |
| ER 11 | 30 | 3 | 0.1 |
| ER 12 | 30 | 10 | 0.005 |
| ER 13 | 10 | 7 | 0.02 |
| AK 05 | 10 | 7 | 0.02 |

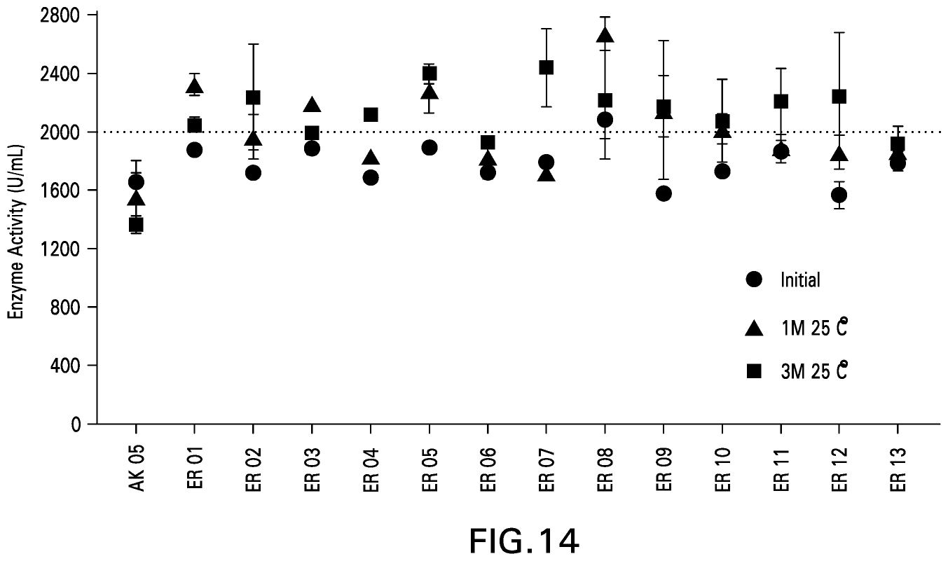

As shown in FIG. 14, there is no observable difference in enzyme activity as a function of excipient concentration or duration of incubation. Test co-formulations of pembrolizumab and PH20 variant fragment 2 that were stored at 25 °C for up to three months show retained activity compared to samples of PH20 variant fragment 2 alone (AK 05), demonstrating stabilization of the enzyme in the presence of pembrolizumab.

### Example 8: Evaluation of the Stability of Recombinant Human Hyaluronidase PH20 Variant Fragment 2 when Incubated at Varying Ratios with Pembrolizumab

Test formulations of pembrolizumab and PH20 variant fragment 2 were prepared with the compositions outlined in Table 17 that differ in their antibody:enzyme ratio. All test formulations were prepared in 0.02% polysorbate 80, 7% sucrose, 10 mM Methionine in 10 mM Histidine buffer at pH 5.5. Samples were incubated at 25 °C for up to three months to monitor the impact of pembrolizumab:PH20 variant fragment 2 ratios on enzyme activity.

TABLE 17
Pembrolizumab + PH20 Variant Fragment
2 Formulations to Explore Impact of Antibody:Enzyme
ratio on PH20 Variant Fragment 2 Activity

| | Pembrolizumab | PH20 Variant Fragment 2 | |
|---|---|---|---|
| Sample | Concentration (mg/mL) | Concentration (Units/mL) | |
| AK 05 | 0 | 0.014 | mg/mL |
| ER 14 | 25 | 0.0009 | mg/mL |
| ER 15 | 175 | 0.0009 | mg/mL |
| ER 16 | 165 | 0.014 | mg/mL |
| ER 17 | 175 | 0.050 | mg/mL |
| ER 18 | 25 | 0.050 | mg/mL |
| ER 19 | 165 | 0.050 | mg/mL |
| ER 20 | 175 | 0.014 | mg/mL |
| ER 21 | 165 | 0.0009 | mg/mL |
| ER 22 | 25 | 0.014 | mg/mL |
| ER 23 | 130 | 0.024 | mg/mL |
| ER 24 | 130 | 0.018 | mg/mL |
| ER 25 | 130 | 0.030 | mg/mL |
| ER 26 | 50 | 0.014 | mg/mL |
| ER 27 | 75 | 0.014 | mg/mL |
| ER 28 | 100 | 0.014 | mg/mL |

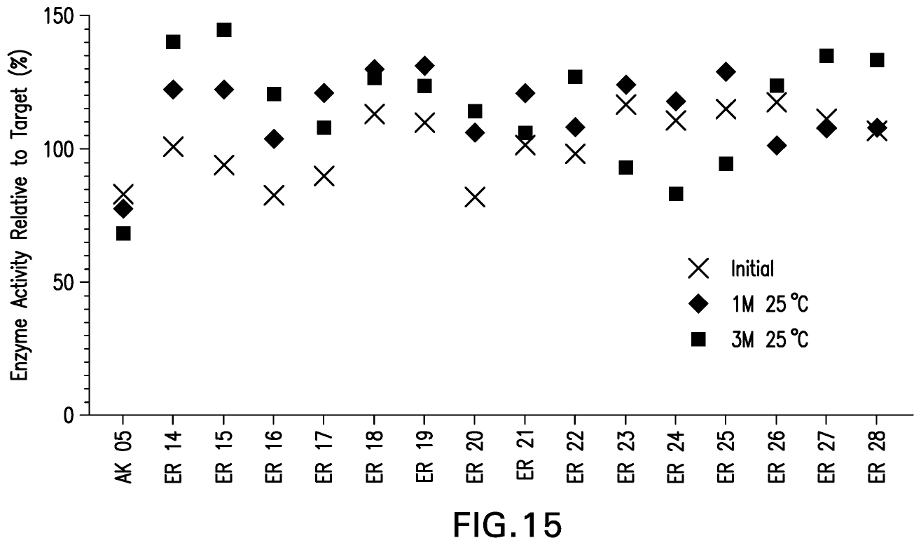

Due to the range of PH20 Variant Fragment 2 concentrations, FIG. 15 displays enzyme activity relative to each sample's target activity level. As shown by the data, enzyme activity for samples with 0.0009-0.05 mg/ml of PH20 Variant Fragment 2 is retained near target after three months of incubation at 25 °C when in the presence of pembrolizumab at 25-175 mg/ml in contrast to samples prepared with PH20 Variant Fragment 2 alone (AK05).

### Example 9: Evaluation of the Stability of Recombinant Human Hyaluronidase PH20 Variant Fragment 2 with Pembrolizumab Under Thermal Stress

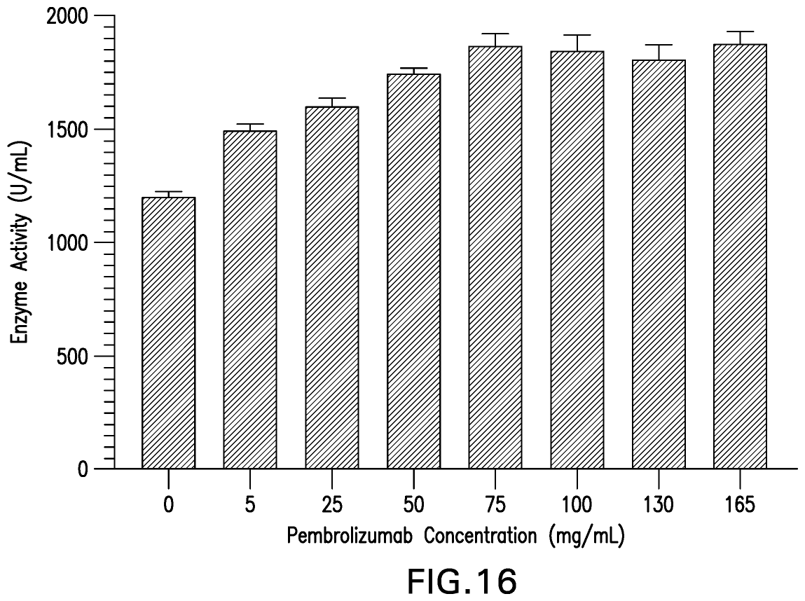

Co-formulated samples were prepared across a range of Pembrolizumab concentrations (5 mg/mL-165 mg/mL), 2000 Units/mL Recombinant Human Hyaluronidase PH20 variant fragment 2 in 7% sucrose, 10 mM methionine in 10 mM Histidine buffer at pH 5.5. Activity was measured after each sample was incubated at 35 °C for 1 week (FIG. 16). The data indicates at concentrations of 5-165 mg/mL Pembrolizumab, there was enhanced PH20 Variant Fragment 2 enzyme activity and stability after thermal stress in the presence of Pembrolizumab compared to samples of PH20 Variant Fragment 2 alone. In addition, retention of PH20 Variant Fragment 2 activity upon thermal stress displays a dependence on Pembrolizumab concentration. The data indicates that at concentrations equal or greater than 75 mg/mL Pembrolizumab, the enhancement of PH20 Variant Fragment 2 enzyme activity was unexpectedly higher compared to lower concentrations of Pembrolizumab.

### Example 10: Impact of pH on the Stability of Recombinant Human Hyaluronidase PH20 Variant Fragment 2 with Pembrolizumab

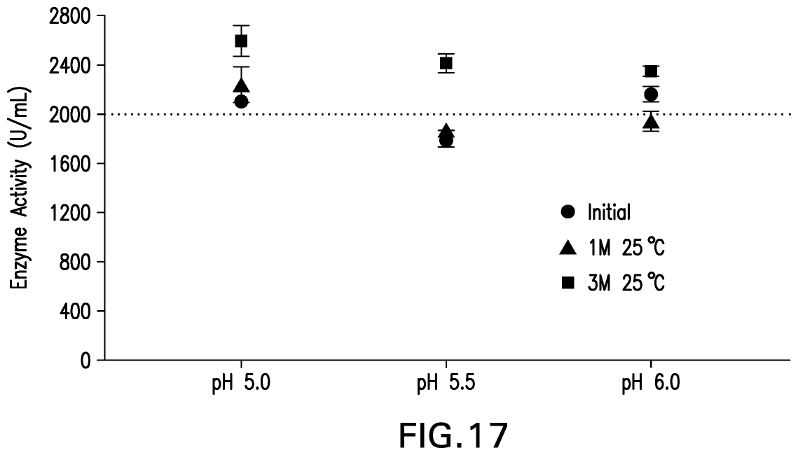

Test formulations of pembrolizumab (165 mg/mL) and PH20 variant fragment 2 (2000 Units/mL) in 10 mM histidine, 10 mM methionine, 7% w/v sucrose, 0.02% w/v PS-80 were prepared at pH 5.0, pH 5.5 and pH 6.0. Samples were incubated at 25 °C for up to three months to monitor the impact of formulation pH on PH20 Variant Fragment 2 stability. FIG. 17 shows that PH20 Variant Fragment 2 enzyme activity is comparable across the pH range studied and maintains activity after incubation for three months at 25 °C.

1-73. (canceled)

74. A formulation comprising:
a) about 50 mg/mL to about 175 mg/mL of pembrolizumab;
b) about 0.0009 mg/ml to about 0.050 mg/ml, about 150 to about 5000 U/ml, about 1000 to about 6000 U/ml of a PH20 variant fragment;
wherein the amino acid sequence of the PH20 variant fragment is as set forth in SEQ ID NO:23;
and means for retaining the enzymatic activity of the PH20 variant fragment after storage at 25 °C for 3 months.

75. A formulation comprising:
a) about 50 mg/mL to about 175 mg/mL of pembrolizumab;
b) about 0.0009 mg/ml to about 0.050 mg/ml, about 150 to about 5000 U/ml, about 1000 to about 6000 U/ml of a PH20 variant fragment;
wherein the amino acid sequence of the PH20 variant fragment is as set forth in SEQ ID NO:23;
and means for retaining the enzymatic activity of the PH20 variant fragment at 5 °C for 6 months or 3 months.

76. A formulation comprising:
a) about 50 mg/mL to about 175 mg/mL of pembrolizumab;
b) about 0.0009 mg/ml to about 0.050 mg/ml, about 150 to about 5000 U/ml, about 1000 to about 6000 U/ml of a PH20 variant fragment;
wherein the amino acid sequence of the PH20 variant fragment is as set forth in SEQ ID NO:23;
and means for enhancing the enzymatic activity of the PH20 variant fragment compared to the formulation without pembrolizumab after storage at 35 °C for 1 week.

77. The formulation of claim 74, wherein pembrolizumab is at about 75 mg/mL to about 175 mg/mL.

78. The formulation of claim 75, wherein pembrolizumab is at about 75 mg/mL to about 175 mg/mL.

79. The formulation of claim 76, wherein pembrolizumab is at about 75 mg/mL to about 175 mg/mL.

80. The formulation of claim 74, wherein pembrolizumab is at about 100 mg/mL to about 165 mg/mL.

81. The formulation of claim 75, wherein pembrolizumab is at about 100 mg/mL to about 165 mg/mL.

82. The formulation of claim 76, wherein pembrolizumab is at about 100 mg/mL to about 165 mg/mL.

83. The formulation of claim 74, wherein pembrolizumab is at about 165 mg/mL to about 170 mg/mL.

84. The formulation of claim 75, wherein pembrolizumab is at about 165 mg/mL to about 170 mg/mL.

85. The formulation of claim 76, wherein pembrolizumab is at about 165 mg/mL to about 170 mg/mL.

86. The formulation of claim 74, wherein the PH20 variant fragment is at about 1000 to about 6000 U/ml.

87. The formulation of claim 75, wherein the PH20 variant fragment is at about 1000 to about 6000 U/ml.

88. The formulation of claim 76, wherein the PH20 variant fragment is at about 1000 to about 6000 U/ml.

89. The formulation of claim 77, wherein the PH20 variant fragment is at about 1000 to about 6000 U/ml.

90. The formulation of claim 78, wherein the PH20 variant fragment is at about 1000 to about 6000 U/ml.

91. The formulation of claim 79, wherein the PH20 variant fragment is at about 1000 to about 6000 U/ml.

92. The formulation of claim 80, wherein the PH20 variant fragment is at about 1000 to about 6000 U/ml.

93. The formulation of claim 81, wherein the PH20 variant fragment is at about 1000 to about 6000 U/ml.


SEQUENCE LISTING (23 sequences)

SEQ ID NO: 1 - protein, 15 aa, synthetic construct
  REGION 1..15: note = source = /note="Description of Artificial Sequence: Synthetic peptide"
RASKGVSTSGYSYLH

SEQ ID NO: 2 - protein, 7 aa, synthetic construct
  REGION 1..7: note = source = /note="Description of Artificial Sequence: Synthetic peptide"
LASYLES

SEQ ID NO: 3 - protein, 9 aa, synthetic construct
  REGION 1..9: note = source = /note="Description of Artificial Sequence: Synthetic peptide"
QHSRDLPLT

SEQ ID NO: 4 - protein, 111 aa, synthetic construct [sequence omitted]

SEQ ID NO: 5 - protein, 218 aa, synthetic construct
  REGION 1..218: note = source = /note="Description of Artificial Sequence: Synthetic polypeptide"
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
  REGION 1..5: note = source = /note="Description of Artificial Sequence: Synthetic peptide"
NYYMY

SEQ ID NO: 7 - protein, 17 aa, synthetic construct
  REGION 1..17: note = source = /note="Description of Artificial Sequence: Synthetic peptide"
GINPSNGGTNFNEKFKN

SEQ ID NO: 8 - protein, 11 aa, synthetic construct
  REGION 1..11: note = source = /note="Description of Artificial Sequence: Synthetic peptide"
RDYRFDMGFDY

SEQ ID NO: 9 - protein, 120 aa, synthetic construct [sequence omitted]

SEQ ID NO: 10 - protein, 447 aa, synthetic construct
  REGION 1..447: note = source = /note="Description of Artificial Sequence: Synthetic polypeptide"
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, synthetic construct
  REGION 1..11: note = source = /note="Description of Artificial Sequence: Synthetic peptide"
RASQSVSSYLA

SEQ ID NO: 12 - protein, 7 aa, synthetic construct
  REGION 1..7: note = source = /note="Description of Artificial Sequence: Synthetic peptide"
DASNRAT

SEQ ID NO: 13 - protein, 9 aa, synthetic construct
  REGION 1..9: note = source = /note="Description of Artificial Sequence: Synthetic peptide"
QQSSNWPRT

SEQ ID NO: 14 - protein, 107 aa, synthetic construct [sequence omitted]

SEQ ID NO: 15 - protein, 214 aa, synthetic construct
  REGION 1..214: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
  REGION 1..5: note = source = /note="Description of Artificial Sequence: Synthetic peptide"
NSGMH

SEQ ID NO: 17 - protein, 17 aa, synthetic construct
  REGION 1..17: note = source = /note="Description of Artificial Sequence: Synthetic peptide"
VIWYDGSKRYYADSVKG

SEQ ID NO: 18 - protein, 4 aa, synthetic construct
  REGION 1..4: note = source = /note="Description of Artificial Sequence: Synthetic peptide"
NDDY

SEQ ID NO: 19 - protein, 113 aa, synthetic construct [sequence omitted]

SEQ ID NO: 20 - protein, 440 aa, synthetic construct
  REGION 1..440: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 22 - protein, 455 aa, synthetic construct
  REGION 1..455: note = source = /note="Description of Artificial Sequence: Synthetic polypeptide"
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 23 - protein, 431 aa, synthetic construct
  REGION 1..431: note = source = /note="Description of Artificial Sequence: Synthetic polypeptide"
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF